SEQUENCE LISTING (10 sequences)

SEQ ID NO: 1 - DNA, 20 bp, synthetic construct
  source 1..20: mol_type = other DNA
gagcagtcccctccctagga

SEQ ID NO: 2 - DNA, 26 bp, synthetic construct
  source 1..26: mol_type = other DNA
ggtttgatgttactgttgcttgattt

SEQ ID NO: 3 - DNA, 19 bp, synthetic construct
  source 1..19: mol_type = other DNA
cggaaacgacgctctcatg

SEQ ID NO: 4 - DNA, 18 bp, synthetic construct
  source 1..18: mol_type = other DNA
ccaggctgaattccccat

SEQ ID NO: 5 - DNA, 20 bp, synthetic construct
  source 1..20: mol_type = other DNA
cgtctgaactatcctgcccg

SEQ ID NO: 6 - DNA, 20 bp, synthetic construct
  source 1..20: mol_type = other DNA
tggtaagggagggatcgttg

SEQ ID NO: 7 - DNA, 23 bp, synthetic construct
  source 1..23: mol_type = other DNA
tttttgtgtgctctcccaggtct

SEQ ID NO: 8 - DNA, 19 bp, synthetic construct
  source 1..19: mol_type = other DNA
tggtcactggttggttggc

SEQ ID NO: 9 - DNA, 22 bp, synthetic construct
  source 1..22: mol_type = other DNA
cgaaaccacataaatcaagccc

SEQ ID NO: 10 - DNA, 22 bp, synthetic construct
  source 1..22: mol_type = other DNA
ctctcttctgggtttattcaga

(19) United States
(12) Patent Application Publication
(10) Pub. No.: US 2025/0381273 A1
(43) Pub. Date: Dec. 18, 2025

(54) Augmenting Mitochondria in Immune Cells for Improved Cancer Immunotherapy

(71) Applicants: LEIBNIZ-INSTITUT FÜR IMMUNTHERAPIE (LIT), Regensburg (DE); The Brigham and Women's Hospital Inc., Boston, MA (US); The United States of America, as Represented by the Secretary, Department of Health, Bethesda, MD (US)

(72) Inventors: Luca GATTINONI, Regensburg (DE); Jeremy BALDWIN, Queensland (AU); Jessica FIORAVANTI, Las Palmas de Gran Canaria (ES); Shiladitya SENGUPTA, Boston, MA (US); Tanmoy SAHA, Boston, MA (US)

(21) Appl. No.: 18/878,307

(22) PCT Filed: Jun. 28, 2023
(86) PCT No.: PCT/EP2023/067747

§ 371 Date: Dec. 23, 2024

Related U.S. Application Data
(60) Provisional application No. 63/356,456, filed on Jun. 28, 2022.

(51) Int. Cl. A61K 40/11; A61K 40/31; A61K 40/42; A61P 35/00; C07K 16/28; C12N 5/0783

(52) U.S. Cl. CPC A61K 40/11; A61K 40/31; A61K 40/4211; A61P 35/00; C07K 16/2803; C12N 5/0636; C12N 2502/1394; C12N 2510/00

(57) ABSTRACT

The present invention relates to compositions and methods in the context of mitochondrial transfer. Disclosed herein are methods that enable the efficient transfer of mitochondria from a donor cell to a recipient cell. The mitochondria-augmented cells are useful in the treatment of diseases and disorders, such as cancer. The present invention also relates to the molecular machinery involved in mitochondrial transfer.

### STATEMENT REGARDING FUNDING

This invention was made with government support under grant number 5U01CA214411-04 awarded by the National Institutes of Health National Cancer Institute. The government has certain rights in the invention. This invention was also made with support from a CRI grant under the Clinic & Laboratory Integration Program (grant CRI3201).

### FILED OF THE INVENTION

The present invention relates to compositions and methods in the context of mitochondrial transfer. Disclosed herein are methods that enable the efficient transfer of mitochondria from a donor cell to a recipient cell. The mitochondria-augmented cells are useful in the treatment of diseases and disorders, such as cancer. The present invention also relates to the molecular machinery involved in mitochondrial transfer.

### BACKGROUND OF THE INVENTION

Adoptive T cell therapies have proven powerful against hematologic malignancies, but efficacy against solid tumor entities is limited. A major hurdle faced by transferred T cells is to overcome the hostile tumor microenvironment, which disrupts normal mitochondrial activity, driving T cell exhaustion. Ultimately, impaired mitochondrial fitness orchestrates transcriptional and epigenetic programs associated with terminal exhaustion, leading to defective antitumor T cell responses and cancer immune evasion. Thus, strategies to boost mitochondrial function in infused T cells are highly sought after. Previous preclinical attempts include leveraging intrinsic T cell properties, such as the generation or selection of T cell subsets with higher mitochondrial fitness, and active intervention strategies, such as genetic engineering of drivers of mitochondrial biogenesis, or the administration of antioxidants during T cell manufacturing to protect mitochondrial integrity. However, these approaches in general narrowly focus on single targets and are largely ineffective if T cells contain mitochondria that are already dysfunctional or have damaged mitochondrial DNA (mtDNA).

In recent years, intercellular transfer of mitochondria has been described, reflecting the evolutionary history of mitochondria as endosymbionts. Mitochondrial transfer has been shown to aid the repair of damaged cells (Proc Natl Acad Sci U S A (2006) 103, 1283-1288; Nature Medicine (2012) 18, 759-765), but also to be exploited by tumor cells, which hijack mitochondria from tumor-infiltrating lymphocytes (Nature Nanotechnology (2022) 17, 98-106) and stromal cells to support their growth (Blood (2017) 130, 1649-1660; Blood (2019) 134, 1415-1429). Several mechanisms of mitochondrial transfer have been described, including trafficking through gap junctions and extrusion of microvesicle-embedded or free-floating mitochondria (Signal Transduction and Targeted Therapy (2021) 6, 65). However, one of the most predominant routes of mitochondrial transfer are tunneling nanotubes (TNT). TNTs are F-actin-supported membrane protrusions that can traverse vast distances to bridge cells enabling the exchange of cytoplasmic factors and organelles between connected cells (Nature Medicine (2012) 18, 759-765; Science (2004) 303, 1007-1010).

The present invention leverages mitochondrial transfer from bone marrow stromal cells (BMSCs) to boost CD8+ T cell bioenergetic capacity, resistance to exhaustion, and antitumor efficacy. TNTs enable effective mitochondrial transfer from BMSCs to T cells, providing the basis for a new technology platform to potentiate the metabolic fitness and antitumor function of T cells for adoptive immunotherapy.

### SUMMARY OF THE INVENTION

The present disclosure relates to a method of augmenting mitochondria in CD8-positive T cells by culturing said CD8-positive T cells with mitochondria donor cells.

The present disclosure relates to a method of augmenting mitochondria in mammalian CD8-positive T cells by culturing said CD8-positive T cells with mitochondria donor cells.

The present disclosure relates to a method of augmenting mitochondria in mammalian CD8-positive T cells by culturing said mammalian CD8-positive T cells with mitochondria donor cells, wherein said mitochondria donor cell is a hematopoietic cell or a stem cell.

The present disclosure relates to a method of augmenting mitochondria in mammalian CD8-positive T cells by culturing said mammalian CD8-positive T cells with mitochondria donor cells, wherein said mitochondria donor cell is a bone marrow stromal cell or a mesenchymal stem/stromal cell.

The present disclosure relates to a method of augmenting mitochondria in mammalian CD8-positive T cells by culturing said mammalian CD8-positive T cells with mitochondria donor cells, wherein said mammalian CD8-positive T cells and/or said mitochondria donor cells are Talin-2 positive.

The present disclosure relates to a method of augmenting mitochondria in mammalian CD8-positive T cells by culturing said mammalian CD8-positive T cells with mitochondria donor cells, wherein said mammalian CD8-positive T cells and/or said mitochondria donor cells are engineered to express Talin-2.

The present disclosure also relates to a mitochondria-augmented mammalian CD8-positive T cell.

The present disclosure also relates to a mitochondria-augmented mammalian CD8-positive T cell obtained by any of the methods disclosed herein.

The present disclosure also relates to a mitochondria-augmented mammalian CD8-positive T cell obtained by any of the methods disclosed herein for use in the treatment of cancer.

The present disclosure also relates to a mitochondria-augmented mammalian CD8-positive T cell obtained by any of the methods disclosed herein for use in the treatment of cancer, wherein said cancer is a solid cancer.

The present disclosure also relates to a mitochondria-augmented mammalian CD8-positive T cell obtained by any of the methods disclosed herein for use in the treatment of cancer, wherein said cancer is a hematological cancer.

The present disclosure also relates to a mitochondria-augmented mammalian CD8-positive T cell obtained by any of the methods disclosed herein for use in enhancing CD8-positive T cell antitumor immunity.

The present disclosure also relates to a mitochondria-augmented mammalian CD8-positive T cell obtained by any of the methods disclosed herein for any of aforementioned used, wherein said treatment additionally comprises a immune checkpoint inhibitor. In certain embodiments said immune checkpoint inhibitor is selected from the group consisting of anti-CTLA4 antibodies, anti-PD-1 antibodies, anti-PD-LI antibodies, anti-PD-L2 antibodies anti-TIM-3 antibodies, anti-LAG3 antibodies, anti-B7H3 antibodies, anti-B7H4 antibodies, anti-BTLA antibodies, and anti-B7H6 antibodies.

### FIGURE LEGENDS

FIG. 1: Intercellular nanotubes enable mitochondrial trafficking from BMSCs to CD8+ T cells. (A-D) FESEM images showing nanotubes (yellow arrows) between BMSCs and CD8+ T cells in human (A, B) and mouse (C, D) co-cultures. The images show thin (A), thick (B), and branched (B1) nanotubes.

FIG. 2: Intercellular nanotubes enable mitochondrial trafficking from BMSCs to CD8+ T cells. Bar graphs showing the number of nanotubes between BMSCs and CD8+ T cells (A), and the distribution of lengths (B) and widths (C) of nanotubes connecting the BMSC and CD8+ T cells, as calculated from the FESEM images. Data shown are mean±s.e.m.

FIG. 3: Intercellular nanotubes enable mitochondrial trafficking from BMSCs to CD8+ T cells. Confocal microscopy images of nanotube (arrows) formation between Mito-DsRed BMSCs and CD8+ T cells in human (A) and mouse (B) co-cultures. Transfer of mitochondria has been observed inside the nanotube (arrows). Co-cultures were fixed after 24 hrs and stained with phalloidin green and DAPI. Co-localization of DAPI and DsRed signals within the nanotube (A) indicates the trafficking of intact mitochondria from the BMSCs to CD8+ T cells.

FIG. 4: Cartoon depicting the transwell coculture system designed to promote mitochondrial transfer from Mito-DsRed BMSCs to CD8+ T cells.

FIG. 5: Establishment and validation of mitochondrial transfer as technology platform. (A) Percentage of mouse CD8+DsRed+T cells 48 hrs after co-culture with Mito-DsRed BMSCs. Data shown are mean±s.e.m., n=19 independent co-culture experiments. (B) Flow cytometry plots of mouse CD8+ T cells 48 hrs after co-culture with Mito-DsRed BMSCs before (left) and after (right) sorting based on DsRed signal. Numbers indicate percentage after gatingon live lymphocytes.

FIG. 6: Establishment and validation of mitochondrial transfer as technology platform. (A) Representative confocal microscopy image showing FACS-sorted mouse CD8+ T cells that have received donor-labeled mitochondria from Mito-DsRed BMSCs. MitoTracker Deep Red FM was used to label total mitochondria after sorting. (B) Correlative confocal and transmission electron microscopy image of FACS-sorted mouse CD8+ T cells that have received donor-labeled mitochondria from MitoDsRed BMSCs. Overlay of nucleus (DAPI) and acquired mitochondria (DsRed) with the electron micrograph of the same section. B1,2: electronmicrograph alone of transferred mitochondria.

FIG. 7: Establishment and validation of mitochondrial transfer as technology platform. Percent increase in mtDNA (as measured by mt-Co2 gene normalized to nuclear App gene) of mouse Mito+ cells relative Mito− (n=7).

FIG. 8: Establishment and validation of mitochondrial transfer as technology platform. Restriction enzyme analysis of Mito+ and Mito− BALB/c CD8+ T cells after co-culture with C57BL/6-derived Mito-DsRed BMSCs. C57BL/6 cells have a single nucleotide polymorphism at A9348 in the mt-Co3 gene that creates an Aspl restriction site.

FIG. 9: Establishment and validation of mitochondrial transfer as technology platform. (A) Oxygen consumption rates (OCR) of FACS-sorted Mito+ and Mito− mouse CD8+ T cells after co-culture with Mito-DsRed BMSCs that were left untreated or pre-treated with 200 ng/ml Ethidium bromide (EtBr) in DMEM complete medium supplemented with 50μg/ml uridine to render donor mitochondria dysfunctional. CD8+ T cells monocultured (CD8 mono) were included as additional control. Data were obtained under basal culture conditions and in response to the indicated molecules. (B) Basal respiration and (C) spare respiratory capacity (SRC) (n=6-12, 2-4 technical replicates per 3-time points). *P<0.05; **P<0.01; ***P<0.001; ***P<0.0001 (one-way ANOVA with Dunnett's multiple comparison test).

FIG. 10: Volcano plot showing changes in gene expression between Mito+ and Mito− human CD8+ T cells. Gene expression was evaluated by RNA-seq on Mito+ and Mito− cells FACS-sorted after 48 hrs co-culture with human Mito-DsRed BMSCs (n=3 healthy donors), (dashed line, P adj=0.05)).

FIG. 11: Mitochondrial transfer from BMSCs to T cells is TLN2-dependent. (A) TLN2 levels in CD8+ T cells and BMSCs following nucleofection of ribonucleoprotein (RNP) complexes targeting TLN2 or CD2, assessed by immunoblot. (B) Percentage of DsRed+CD8+ T cells after BMSCs-CD8+ T cell co-cultures in which TLN2 was deleted in the indicated cell type. Data are shown as mean±s.e.m. relative to control co-cultures in which CD2 was deleted. *P<0.05 (unpaired two-tailed Student's t-test).

FIG. 12: Mitochondrial transfer enhances CD8+ T cell antitumor immunity against solid tumors. Tumorsize (A, mean±s.e.m.) and survival curve (B) of sublethally irradiated B16KVP tumor-bearing Ly5.2+ mice receiving 1.5×10⁵ Mito+ or Mito− pmel-1 Ly5.1+CD8+ T cells generated as in FIG. 2A in conjunction with recombinant human IL-2 (n=5 mice/group). No Tx, no treatment (n=4 mice).

FIG. 13: Mitochondrial transfer enhances CD8+ T cell antitumor immunity against solid tumors. Flow cytometry analysis of mouse CD8+ T cells after a 48 h co-culture with Mito-DsRed BMSCs. Right panel shows the expression of CD44 and CD62L non overlaid Mito+ and Mito−.

FIG. 14: Mitochondrial transfer enhances CD8+ T cell antitumor immunity against solid tumors. Tumor size (A, mean±s.e.m.) and survival curve (B) of sublethally irradiated NCG mice bearing B16 tumors after treatment with 2.5×10⁵ Mito+ and Mito− pmel-1 Ly5.1+CD8+ T cells with recombinant human IL-2 (n=8 or 9 mice/group). No Tx, no treatment (n=5mice). *P<0.05; (Wilcoxon rank sum test, A; log-rank [Mantel-Cox] test, B) **P<0.01 (log-rank [Mantel-Cox] test).

FIG. 15: Mitochondrial transfer enhances CD8+ T cell antitumor immunity against solid tumors. 2.5×10⁵ Mito+ and Mito− pmel-1 Ly5.1+CD8+ T cells were adoptively transferred into sublethally irradiated Ly5.2+ mice bearing B16 tumors in conjunction with recombinant human IL-2 (A-C) Flow cytometry plot (A), frequency (B) and absolute numbers (C) of |Mito+ and Mito− pmel-1 Ly5.1+CD8+ T cells in the spleen 7 d after treatment. *P<0.05 (unpaired two-tailed Student's t-test).

FIG. 16: Mitochondrial transfer enhances CD8+ T cell antitumor immunity against solid tumors. Numbers of pmel-1 Ly5.1+CD8+ T cells per mg of tumor tissue, 7 d after treatment. Data shown as mean±s.e.m. (n=4 and 5).

FIG. 17: Mitochondrial transfer enhances CD8+ T cell antitumor immunity against solid tumors. Flow cytometry plots (A) and frequencies (B, C) of Mito+ and Mito− pmel-1 Ly5.1+CD8+ T cells expressing high (B) and intermediate (C) levels of PD1 and LAG3 7 d after treatment as in A, B. Data shown are mean±s.e.m. (n=4 and 5). *P<0.05 (unpaired two-tailed Student's t-test).

FIG. 18: Mitochondrial transfer enhances CD8+ T cell antitumor immunity against solid tumors. 2.5×10⁵ Mito+ and Mito− pmel-1 Ly5.1+CD8+ T cells were adoptively transferred into sublethally irradiated Ly5.2+ mice bearing B16 tumors in conjunction with recombinant human IL-2 (AC). Flow cytometry plot (A) and frequency (B,C) of intratumoral Mito+ and Mito− pmel-1 Ly5.1+CD8+ T cells expressing high (B) and intermediate (C) levels of PD-1 and TIGIT 7 d after tumor treatment.

FIG. 19: Mitochondrial transfer enhances CD8+ T cell antitumor immunity against solid tumors. Flow cytometry plots (A) and frequencies (B, C) of Mito+ and Mito− pmel-1 Ly5.1+CD8+ T cells expressing the indicated combinations of PD1 and Gzmb (B, C) 7 d after treatment. Data shown are mean±s.e.m. (n=4 and 5). *P<0.05 (unpaired two-tailed Student's t-test).

FIG. 20: Mitochondrial transfer enhances human CD19-CAR CD8+ T cell antitumor immunity. Cytotoxicity assay using CD19-CAR Mito− or Mito+ cells after co-culture with Mito-DsRed BMSCs that were left untreated or pre-treated Ethidium bromide (EtBr) to render donor mitochondria dysfunctional. Green calibrated Unit (GCU) per mm2/image at indicated time points after co-culture. Data are shown as mean±s.e.m. (n=3 technical replicates per group).

FIG. 21: Mitochondrial transfer enhances human CD19-CAR CD8+ T cell antitumor immunity. (A) Numbers of circulating NALM6-GL cells per 50 μl of blood 7 d after transfer of 1.25×10⁵ CD19-CAR Mito− or Mito+ cells or CD8 monocultured in conjunction with recombinant human IL-15 into sublethally irradiated NXG mice bearing NALM6-GL leukemia. (B) Survival of NALM6-GL-bearing NXG mice treated as in (A) (n=4 or 6 mice/group). No Tx, no treatment (n=6). *P<0.05 (Kruskal-Wallis test, (A), log-rank [Mantel-Cox] test, (B)).

### DETAILED DESCRIPTION OF THE INVENTION

Organelle medicine, or organelle transplantation, is an emerging research area, wherein similar to traditional organ transplants in patients, organelles are transferred to recipient cells to improve cellular function. Mitochondria transfer is one form of organelle transplantation, but its application to T cell therapy has yet to be elucidated.

Preclinical and clinical studies of adoptive T cell therapy have shown that the metabolic qualities of the infusion products, and in particular their mitochondria function, are critical determinants of patients' outcomes. Unfortunately, patient or donor T cell mitochondria can become damaged and dysfunctional, impairing their capacity to energetically sustain the fight against cancer cells upon transfer of these ‘living drugs’ (J Exp Clin Cancer Res (2022) 41, 227). Indeed, mitochondrial DNA (mtDNA) is up to 10 times more prone to accumulate damage than nuclear DNA (Biochimica et Biophysica Acta (BBA)-Molecular Basis of Disease (1995) 1271, 177-189). Approximately 60% of cancer cases occur in patients aged 65 and above, increasing the likelihood of accumulated mtDNA mutations. Moreover, mitochondrial biomass and activity in T cells sharply decrease with age. Prior systemic treatments, including chemo- and radiotherapy can also have detrimental effects on mitochondrial function of patients' T cells (Br J Cancer (2007) 97, 105-111). Thus, the ability to transplant healthy mitochondria can have a profound impact on several cancer immunotherapy platforms, such as those relying on autologous T cell sources and in particular on tumor-infiltrating lymphocytes whose mitochondria have been damaged by the hostile tumor microenvironment.

Our results provide proof of concept that BMSC mitochondria transfer can be successfully utilized to enhance the antitumor efficacy of both mouse and human CD8+ T cells using different tumor-redirecting constructs (TCR/CAR) in different in vivo settings (mouse syngeneic/humanized xenograft) against both liquid and solid tumors. Mitochondrial transfer from donor BMSCs enabled antitumor CD8+ T cells to expand robustly, infiltrate the tumor mass more efficiently, resist exhaustion, and differentiate into potent cytotoxic effector cells. Interestingly, a high portion of cells that were prone to exhaustion in the group that received BMSC mitochondria showed reduced expression levels of PD1, LAG3, and TIGIT. As this cell population can be rescued more efficiently by PD-1: PD-L1 blockade compared to terminally exhausted PD1^{hi}LAG3^{hi}TIGIT^{hi}, it may be beneficial in the future to couple mitochondria-boosted T cell therapies with immune checkpoint inhibitors.

The present disclosure discloses immune cells that are loaded with exogenous mitochondria by culturing them with donor cells, such as hematopoietic cells or stem cell s. The T cells form nanotubes with the donor cells, and it is demonstrated that the mitochondria from the donor cells are trafficked to the T cells through these nanotubes. Such mitochondria augmented immune cells then exert greater antitumor effect. This has significant impact on immunotherapy, including on CAR-T cells.

It is believed that this is the first study to describe the transfer of mitochondria from stem cells, such as mesenchymal stem/stromal cells, (donor cell) to T cells, such as CD8-positive T cells, which supercharges the T cells to exert a greater antitumor effect.

### Definitions

The term “augmenting” as used herein in the context of mitochondrial transfer refers to a method or procedure in which mitochondria are transferred from a donor cell to a recipient cell, such that the recipient cell contains a higher number of mitochondria after such transfer as compared to prior of such transfer.

The term “CD8-positive T cells” refers to T cells that are positive for the CD8 marker. CD8-positive T cells are involved in the cytotoxic immune response.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential.

The term “hematopoietic cell” refers to a cell that arises from a hematopoietic stem cell. This includes, but is not limited to, myeloid progenitor cells, lymphoid progenitor cells, megakaryocytes, erythrocytes, mast cells, myeloblasts, basophils, neutrophils, eosinophils, macrophages, thrombocytes, monocytes, natural killer cells, T lymphocytes, B lymphocytes and plasma cells.

The term “bone-marrow stromal cell” as used herein refers to cells present in tissue which is present in bone marrow and has a network structure.

The term “mesenchymal stem/stromal cell” as used herein refers to fibroblast-like cells with multipotent differentiation capacity, such as chondrocytes, osteoblasts, adipocytes, myoblasts, and others.

The term “mammal” or “mammalian” as used herein refers to any animal of the class Mammalia including human, mouse, rat, cat, dog, sheep, rabbit, horse, cow, goat, pig, guinea pig, hamster, chicken, turkey, or non human primates (e.g., Marmoset, Macaque)).

The term “Talin-2” refers to a protein also known as KIAA0320 or WILEQ, UniProt: Q9Y4G6. The term “Talin-2 positive” in the context of a cell refers to a cell which expresses a functional Talin-2 protein.

The term “engineered to express Talin-2” in the context of a cell refers to a cell which is recombinantly engineered to express or overexpressed Talin-2 by any known technology in the art, including but not limited to Crispr/Cas, and other technologies relying on the Crispr/Cas machinery like base editing or prime editing.

The term “T cell receptor” or “TCR” is art recognized and refers to a complex of membrane proteins that participate in the activation of T cells in response to the presentation of antigen. A TCR is responsible for recognizing antigens bound to major histocompatibility complex molecules. A TCR comprises a heterodimer of an alpha and beta chain, although in some cells the TCR comprises gamma and delta chains.

The term “chimeric antigen receptor” or “CAR” is art recognized and refers to a chimeric polypeptide that is designed to include an optional signal peptide, an antigen binding domain, an optional hinge, a transmembrane domain, and one or more intracellular signaling domains.

The term “cancer” as used herein refers to or describes the physiological condition in mammals, in particular humans, which is typically characterized by unregulated cell growth. Examples of cancer include, but are not limited to, carcinoma, lymphoma, blastoma, sarcoma, and leukemia. The term “cancer” includes solid cancers and hematological cancers.

The term “solid cancer” as used herein refers to a cancer that forms a discrete tumor mass, i.e., a solid tumor. Examples of solid cancers within the scope of the present methods include cancers of the bladder, colon, rectum, kidney, prostate, brain, breast, liver, lung, skin (e.g., melanoma), and head and neck.

The term “hematological cancer” as used herein refers to cancers mat occur in cells of the immune system or in blood-forming tissues including bone marrow and which generally do not form solid tumors. Examples of hematologic cancers within the scope of the present methods include leukemia (e.g., acute myeloid leukemia, acute lymphoblastic leukemia, chronic myelogenous leukemia, and chronic lymphocytic leukemia), Hodgkin and non-Hodgkin lymphoma, myeloma, and myelodysplastic syndrome.

The term “immune checkpoint inhibitor” as used herein refers to any compound inhibiting the function of an immune inhibitory checkpoint protein. Inhibition includes reduction of function and full blockade. Immune checkpoint inhibitors include antibodies that specifically recognize immune checkpoint proteins. In some embodiments, the immune checkpoint inhibitor is an antibody selected from the group consisting of anti-CTLA4 antibodies, anti-PD-1 antibodies, anti-PD-L1 antibodies, anti-PD-L2 antibodies anti-TIM-3 antibodies, anti-LAG3 antibodies, anti-B7H3 antibodies, anti-B7H4 antibodies, anti-BTLA antibodies, and anti-B7H6 antibodies.

### Embodiments of the Invention

In certain embodiments the present disclosure relates to a method to transfer mitochondria from a donor cell to a recipient cell. In certain embodiments, said recipient cell is a CD8-positive T cells. In certain embodiments, said recipient cell is a mammalian CD8-positive T cells. Therefore, in certain embodiments the present disclosure relates to a method to transfer mitochondria into CD8-positive T cells by culturing said CD8-positive T cells with mitochondria donor cells. In other embodiments the present disclosure relates to a method to transfer mitochondria into mammalian CD8-positive T cells by culturing said mammalian CD8-positive T cells with mitochondria donor cells.

In certain embodiments, the present disclosure relates to a method of augmenting mitochondria in mammalian CD8-positive T cells by culturing said mammalian CD8-positive T cells with mitochondria donor cells.

The donor cell may be a hematopoietic cell, a stem cell or a bone marrow stromal cell. Preferably said donor cell is a bone marrow stromal cell. Therefore, in certain embodiments the present disclosure relates to a method of augmenting mitochondria in mammalian CD8-positive T cells by culturing said mammalian CD8-positive T cells with hematopoietic cells, stem cells, bone marrow stromal cells or mesenchymal stem/stromal cells. In preferred embodiments, the present disclosure relates to a method of augmenting mitochondria in mammalian CD8-positive T cells by culturing said mammalian CD8-positive T cells with bone marrow stromal cells or mesenchymal stem/stromal cells. In other preferred embodiments, the present disclosure relates to a method of augmenting mitochondria in mammalian CD8-positive T cells by culturing said mammalian CD8-positive T cells with bone marrow stromal cells. In other preferred embodiments, the present disclosure relates to a method of augmenting mitochondria in mammalian CD8-positive T cells by culturing said mammalian CD8-positive T cells with mesenchymal stem/stromal cells.

The present disclosure also shows that an effective transfer of mitochondria from a donor cell to a recipient cell as shown herein is dependent on Talin-2. Donor cell and/or recipient cells may therefore be engineered to express or to overexpress Talin-2. Respective methods to insert genes into cells are known in the art and include technologies like viral and non-viral transduction technologies or gene/genome editing via technologies like CRISPR/Cas, and other technologies relying on the Crispr/Cas machinery like base editing or prime editing.

Therefore in certain embodiments, the present disclosure relates to a method of augmenting mitochondria in recipient cells by culturing said recipient cells with mitochondria donor cells, wherein said donor cells or said recipient cells are Talin-2 positive. In other embodiments, the present disclosure relates to a method of augmenting mitochondria in recipient cells by culturing said recipient cells with mitochondria donor cells, wherein said donor cells or said recipient cells express Talin-2. In other embodiments, the present disclosure relates to a method of augmenting mitochondria in recipient cells by culturing said recipient cells with mitochondria donor cells, wherein said donor cells or said recipient cells overexpress Talin-2. In other embodiments, the present disclosure relates to a method of augmenting mitochondria in recipient cells by culturing said recipient cells with mitochondria donor cells, wherein said donor cells or said recipient cells are engineered to express or to overexpress Talin-2. Preferably said recipient cell is a CD8-positive T cell. Also preferably said donor cell is a hematopoietic cell, a stem cell, a bone marrow stromal cell or a mesenchymal stem/stromal cell.

The therapeutic usefulness of the mitochondria-augmented mammalian CD8-positive T cells is demonstrated in the examples of the present invention. Particularly useful are mitochondria-augmented mammalian CD8-positive T cells which additionally comprise an antigen-specific receptor, such as a T cell receptor (TCR) or a chimeric antigen receptor (CAR).

Therefore in certain embodiments, the present disclosure relates to a method of augmenting mitochondria in recipient cells by culturing said recipient cells with mitochondria donor cells, wherein said recipient cells further comprises an antigen-specific receptor.

In certain embodiments, the present disclosure relates to a method of augmenting mitochondria in recipient cells by culturing said recipient cells with mitochondria donor cells, wherein said recipient cells further comprises an antigen-specific receptor selected from a T cell receptor (TCR) or a chimeric antigen receptor (CAR).

In certain embodiments, the present disclosure relates to a method of augmenting mitochondria in recipient cells by culturing said recipient cells with mitochondria donor cells, wherein said recipient cells further comprises a T cell receptor (TCR).

In certain embodiments, the present disclosure relates to a method of augmenting mitochondria in recipient cells by culturing said recipient cells with mitochondria donor cells, wherein said recipient cells further comprise a chimeric antigen receptor (CAR).

In certain embodiments, said antigen-specific receptor is specific for a cancer antigen.

In certain embodiments, said antigen-specific receptor is specific for gp100 or CD19.

In certain embodiments, the present disclosure relates to a mitochondria-augmented CD8-positive T cell.

In certain embodiments, the present disclosure relates to a mitochondria-augmented mammalian CD8-positive T cell.

In certain embodiments, the present disclosure relates to a mitochondria-augmented CD8-positive T cell obtained by any of the aforementioned methods.

In certain embodiments, the present disclosure relates to a mitochondria-augmented mammalian CD8-positive T cell obtained by any of the aforementioned methods.

In certain embodiments, the present disclosure relates to a mitochondria-augmented mammalian CD8-positive T cells which additionally comprise an antigen-specific receptor.

In certain embodiments, the present disclosure relates to a mitochondria-augmented mammalian CD8-positive T cells which additionally comprise an antigen-specific receptor selected from a T cell receptor (TCR) or a chimeric antigen receptor (CAR).

In certain embodiments, the present disclosure relates to a mitochondria-augmented mammalian CD8-positive T cells which additionally comprise a T cell receptor (TCR).

In certain embodiments, the present disclosure relates to a mitochondria-augmented mammalian CD8-positive T cells which additionally comprise a chimeric antigen receptor (CAR).

In certain embodiments, said antigen-specific receptor is specific for a cancer antigen.

In certain embodiments, said antigen-specific receptor is specific for gp100 or CD19.

As shown in the examples herein, the mitochondria-augmented mammalian CD8-positive T cell have may be used in the treatment of cancer, such as solid cancers or hematological cancers. The mitochondria-augmented mammalian CD8-positive T cell may also be used for enhancing CD8-positive T cell antitumor immunity.

Therefore, in certain embodiments the present disclosure relates to a mitochondria-augmented mammalian CD8-positive T cell prepared by the methods disclosed herein for use in the treatment of cancer. In certain embodiments the present disclosure relates to a mitochondria-augmented mammalian CD8-positive T cell prepared by the methods disclosed herein for use in the treatment of a solid cancer. In certain embodiments the present disclosure relates to a mitochondria-augmented mammalian CD8-positive T cell prepared by the methods disclosed herein for use in the treatment of a hematological cancer. In certain embodiments the present disclosure relates to a mitochondria-augmented mammalian CD8-positive T cell prepared by the methods disclosed herein for use in enhancing CD8-positive T cell antitumor immunity.

In certain embodiments the present disclosure relates to a method of treating a cancer patient, wherein a mitochondria-augmented mammalian CD8-positive T cell prepared by the methods disclosed herein is administered to the patient. In certain embodiments the present disclosure relates to a method of treating a cancer patient, wherein a mitochondria-augmented mammalian CD8-positive T cell prepared by the methods disclosed herein is administered to the patient, and wherein said cancer is a solid cancer. In certain embodiments the present disclosure relates to a method of treating a cancer patient, wherein a mitochondria-augmented mammalian CD8-positive T cell prepared by the methods disclosed herein is administered to the patient, and wherein said cancer is a hematological cancer.

In certain embodiments the present disclosure relates to a method of enhancing CD8-positive T cell antitumor immunity via a mitochondria-augmented mammalian CD8-positive T cell prepared by the methods disclosed herein.

As demonstrated herein, it is also beneficial to combine mitochondria-boosted T cell therapies with immune checkpoint inhibitors. Therefore, in certain embodiments aforementioned treatments are combined with the administration of an immune checkpoint inhibitor. In certain embodiments aforementioned treatments are combined with the administration of a therapeutically effective amount of an immune checkpoint inhibitor.

In certain embodiments the present disclosure relates to a mitochondria-augmented mammalian CD8-positive T cell prepared by the methods disclosed herein and an immune checkpoint inhibitor for use in the treatment of cancer. In certain embodiments the present disclosure relates to a mitochondria-augmented mammalian CD8-positive T cell prepared by the methods disclosed herein and an immune checkpoint inhibitor for use in the treatment of a solid cancer. In certain embodiments the present disclosure relates to a mitochondria-augmented mammalian CD8-positive T cell prepared by the methods disclosed herein and an immune checkpoint inhibitor for use in the treatment of a hematological cancer. In certain embodiments the present disclosure relates to a mitochondria-augmented mammalian CD8-positive T cell prepared by the methods disclosed herein and an immune checkpoint inhibitor for use in enhancing CD8-positive T cell antitumor immunity.

In certain embodiments the present disclosure relates to a method of treating a cancer patient, wherein a mitochondria-augmented mammalian CD8-positive T cell prepared by the methods disclosed herein is administered to the patient in combination with an immune checkpoint inhibitor. In certain embodiments the present disclosure relates to a method of treating a cancer patient, wherein a mitochondria-augmented mammalian CD8-positive T cell prepared by the methods disclosed herein is administered to the patient in combination with an immune checkpoint inhibitor, and wherein said cancer is a solid cancer. In certain embodiments the present disclosure relates to a method of treating a cancer patient, wherein a mitochondria-augmented mammalian CD8-positive T cell prepared by the methods disclosed herein is administered to the patient in combination with an immune checkpoint inhibitor, and wherein said cancer is a hematological cancer.

In certain embodiments the immune checkpoint inhibitor is selected from the group consisting of anti-CTLA4 antibodies, anti-PD-1 antibodies, anti-PD-LI antibodies, anti-PD-L2 antibodies anti-TIM-3 antibodies, anti-LAG3 antibodies, anti-B7H3 antibodies, anti-B7H4 antibodies, anti-BTLA antibodies, and anti-B7H6 antibodies.

### EXAMPLES

### Example 1: Materials and Methods

### Cell Lines

Immortalized BMSCs (SL428, GeneCopoeia) and DM5, a spontaneously immortalized mBMSC line (Stem Cells (2015) 33, 1304-1319) were transduced to express the fluorescent protein DsRed fused with cytochrome c oxidase subunit 8A (COX8A) (Mito-DsRed). Mito-DsRed retrovirus was produced in Platinum-E cells (Cell Biolabs) and 293GP cells (ATCC) were used for the production of Mito-DsRed retroviral vectors. Mouse CD8+ T cells were isolated from either C57BL/6, pmel-1 Ly5.1, or BALB/c mice using a Total CD8+ T cell isolation kit (Stem Cell Technologies). Human CD8+ T cells were isolated from peripheral blood mononuclear cell buffy coats or leukocyte reduction system chambers of healthy donors (NIH, US and Universitätsklinikum Regensburg, Germany) using a Naïve CD8+ T cell isolation kit (Stem Cell Technologies). NALM6 cell line was originally obtained from DSMZ (ACC 128) and transduced with Luciferase-GFP (GL) as previously described (Blood (2011) 118, e112-e117). PG13 expressing CD19-CAR (FMC63-28-ζ) retrovirus was used for transduction of human CD8+ T cells with CD19 CAR as previously described (Journal of immunotherapy (Hagerstown, Md.: 1997) 32, 689). B16KVP melanoma expressing human gp100 was engineered as previously described (JCI Insight (2019) 4 (10): e124405). NALM6-GL and both human and mouse CD8+ T cells were cultured with RPMI complete medium; human BMSCs, PG13 CD19-CAR, B16KVP, Platinum-E, and 293GP cells were cultured with DMEM complete medium; and DM5cell line was cultured with MEM-α complete medium. All cell lines were regularly tested and validated as being mycoplasma free via a PCR-based assay (PromoCell).

### Experimental Animals

C57BL/6 and BALB/c mice ages 6-8 weeks were obtained from Charles River. Immunodeficient NCG and NXG mice ages 6-8 weeks were obtained from Charles River and Janvier Labs, respectively. Pmel-1-Ly5.1 were generated in house breeding at either at animal facilities at the US National Institutes of Health or Universitätsklinikum Regensburg. All mice were housed in a specific pathogen-free facility under standard conditions (12 h light/dark, food and water ad libitum). All mouse experiments were performed in strict accordance with the relevant guidelines and regulations of the University of Regensburg and US National Cancer Institute. All protocols were approved by relevant Animal Care and Use Committee at the US National Institutes of Health and the German authorities.

### Antibodies, Flow Cytometry and Cell Sorting

For cell sorting, antibodies anti-human CD8 (SK1) and anti-mouse CD8 (53-6.7) from BD Biosciences were used together with LIVE/DEAD™ Fixable Far Red Dead Cell Stain (Invitrogen™) unless otherwise stated. For flow cytometry analyses the following antibodies were employed: anti-Sca-1 (D7), anti-CX3CR1 (SA011F11), anti-CD69 (H1.2F3) anti-CD3 (145-2C11), anti-KLRG1 (2F1/KLRG1), anti-CD27 (LG.3A10), anti-CD44 (IM7), anti-CD8a (53-6.7), anti-CD366 (B8.2C12), anti-Granzyme B (GB11), anti-PD-1 (RMPI-30) and anti-LAG-3 (C9B7W) were from Biolegend; anti-CD62L (MEL-14), anti-IL7Rα (SB/199), anti-CD45.2 (104), anti-CD244.2 (2B4), anti-TIGIT (1G9) were from BD Pharmingen; anti-CD122 (TM-Beta) was from BD Biosciences; anti-CD45.1 (A20) was from eBiosciences; and anti-TCF1 (C6309) was from Cell Signaling Technology. TruStain FcX™ used for blocking non-specific binding of immunoglobulin was from Biolegend and LIVE/DEAD™ Fixable Blue Dead Cell Stain was from Thermo Fisher Scientific. Ultracomp eBeads™ Plus (Invitrogen™) were used for compensation. LSR II or BDFortessa and BD FACSymphony (BD Biosciences) were used for flow cytometry acquisition and a FACSAria Fusion (BD Biosciences) or BD Influx (BD Biosciences) were employed for cell sorting. Samples were analyzed with FlowJo software 10.8.2 (BD Biosciences).

### Scanning Electron Microscopy of Nanotubes

Human or mouse BMSCs and CD8+ T cells were co-cultured on 12 mm diameter glass coverslips (VWR) for 24 hrs according to the experimental outline. The sample was fixed by 2.5% glutaraldehyde (Sigma-Aldrich) in 0.1 M sodium cacodylate buffer (Electron Microscopy Sciences). Cells were then washed 3×15 min with 0.1 M sodium cacodylate buffer, post-fixed in 0.1% Osmium tetroxide (OsO4) (Sigma-Aldrich) in water for 1 hr at room temperature (RT) and washed 2×10 min with water before dehydration. The dehydration step was performed as follows: 35% ethanol for 5 min, 50% ethanol for 5 min, 70% ethanol for 10 min, 90% ethanol for 10 min, and 2×100% ethanol for 10 min. Following fixation and dehydration, the coverslips were dried and placed on FESEM stubs for sputter coating by EMS 300T D Dual Head Sputter Coater with Au or Pt/Pd (5 nm). Imaging was acquired on a Zeiss Ultra55 microscope equipped with a Gemini column and SE2 detector. Images were processed in Image J software.

### Fluorescence Microscopy of Nanotubes

Human or mouse BMSCs and CD8+ T cells were co-cultured on 12 mm diameter glass coverslips (VWR). After 24 h, cells were fixed with 4% paraformaldehyde (PFA) (Electron Microscopy Sciences) at room temperature for 2 h. The fixed cells were washed three times with 1× PBS for 20 min. For actin staining, cells were incubated at room temperature for 1 h with 50 μg/ml of Phalloidin AF-488 (Thermo Fisher Scientific) and incubated. For nuclear staining, cells were incubated with Hoechst 33342 (Thermo Fisher Scientific) for 15-20 min. The cells were washed with 1× PBS-T for three more times. The coverslips were mounted on glass slides and images were taken on a Nikon Eclipse Ti camera (Nikon Instruments) with NIS Elements Imaging Software (3.10) or TissueFAXS Plus slide scanner (TissueGnostics USA) equipped with Hamamatsu Orca Flash 4.0 V2 cooled digital CMOS camera. Confocal fluorescence microscopy was done on Zeiss LSM 800, Airyscan Confocal Laser Scanner Microscope with Zen 2.3 software. Post-processing of the images was done either in Image J or Zen lite software.

### Murine Co-Culture System Platform

For murine samples, transwells with 25 mm or 75 mm with 0.4 μm pore size polycarbonate membrane insert (Corning) were used for in vitro co-culture of BMSCs and CD8+ T cells. In brief, mouse CD8+ T cells were isolated from either C57BL/6, pmel-1 Ly5.1, or BALB/c mice using either Total CD8+ T cell isolation kit (Stem Cell Technologies) and activated in tissue culture-treated 24-well plates (Corning) using either anti-mouse CD3e (145.2C11)/CD28 (37.51) both from BD Pharmingen, or for pmel-1 cells only whole splenocytes were isolated and activated using 1 μM human gp10025-33 peptide (Genescript), in RPMI complete media supplemented with rhIL-2 (Proleukin) for 3 days. On the day of co-culture the inserts of the transwells were seeded with either 2×10⁵ (for 25 mm) or 2×10⁶ (for 75 mm) Mito-DsRed labeled mouse DM5 BMSCs in complete MEM-α media. After allowing BMSCs to attach for at least 5 hrs, media in the insert was removed, and pre-activated mouse CD8+ T cells were seeded at a ratio of 1:1 or 3:1 in RPMI complete media. After 24-36 hrs cells were collected for further analysis.

### Human Co-Culture System Platform

For human samples, tissue culture-treated 6-well plate (Corning) coated with collagen solution (Sigma-Aldrich) or pre-coated BioCoat™ 10 mm Petri dishes (Corning) were used for in vitro co-culture of BMSCs and CD8+ T cells. In brief, CD8+ T cells were isolated from buffy coats from healthy donors using a Naive CD8+ T cell isolation kit (Stem Cell Technologies) and activated using 3:1 ratio (beads:cells) of CD3/28 Dynabeads beads (Invitrogen™) in RPMI complete media supplemented with 60 IU/ml of rIL-2 (Proleukin) for 2 days and 3 days further expansion. On the day of co-culture the tissue culture plates were seeded with either 2×10⁵ (for 6-well plate) 2×10⁶ (for 10 mm petri dish) Mito-DsRed labeled human BMSCs in complete DMEM media. After allowing BMSCs to attach for at least 5 hrs, media was removed, and pre-activated human CD8+ T cells were seeded at a ratio of 1:1 or 3:1 in RPMI complete media. After 24-36 hrs cells were collected for further analysis.

### Confocal Imaging of Sorted Cells

Sorted human and mouse Mito+ and Mito− cells (1×10⁵) were stained in 10 nM MitoTracker Deep Red (ThermoFisher Scientific) in PBS for 15 minutes at 37° C. After incubation, cells were washed with completed RPMI media to remove excess dye, and then counterstained with either Alexa-488 conjugated anti-mouse CD8 (53.6.7, Biolegend) or Alexa-488 conjugated anti-human CD8 (SK1, Biolegend), in conjunction with Hoechst 33342 (Invitrogen™, 2 μg/mL dilution) for 20 mins at 4° C. Stained cells were then seeded into 8-well μ-Slide (ibidi Gmbh) coated with Cell-Tak™ (Corning) and visualized using either Stellaris 8 (Leica Microsystems) or SoRa (Nikon) confocal microscope.

### Quantification of mtDNA Content

Quantification of mtDNA was assessed with real-time quantitative PCR. Total DNA was isolated from BMSCs using Quick Extract™ DNA Extaction Solution (Lucigen), according to the manufacturer's protocol. Real-time quantitative PCR was performed in triplicates on 96-well plates (Applied Biosystems). Each PCR reaction (final volume 25 μl) contained 25 ng DNA, 12.5 μl of PowerUp™ SYBR™ Green PCR Master Mix (Applied Biosystems) and 0.5 μM of each forward and reverse primer. MtDNA was quantified using primers specific for the mouse or human MT-CO2 gene and normalized using primers specific for the murine or human reference gene APP. Primers were as follows; mouse mt-Co2-F (GAGCAGTCCCCTCCCTAGGA; (SEQ ID NO. 1)), mouse mt-Co2-R (GGTTTGATGTTACTGTTGCTTGATTT; (SEQ ID NO. 2)), mouse App-F (CGGAAACGACGCTCTCATG; (SEQ ID NO. 3)), mouse App-R (CCAGGCTGAATTCCCCAT; (SEQ ID NO. 4)), human MT-CO2-F (CGTCTGAACTATCCTGCCCG; (SEQ ID NO. 5)), human MT-CO2-R APP-F (TGGTAAGGGAGGGATCGTTG; (SEQ ID NO. 6)), human (TTTTTGTGTGCTCTCCCAGGTCT; (SEQ ID NO. 7)), and human APP-R (TGGTCACTGGTTGGTTGGC; (SEQ ID NO. 8)).

### Generation of BMSC Lines With Dysfunctional Donor Mitochondria

To obtain BMSC with dysfunctional donor mitochondria, BMSCs were cultured in the presence of 50 ng/ml EtBr (human cells) or 200 ng/ml EtBr (mouse cells) for 14 days. EtBr was added to the respective culture medium together with 50 μg/ml uridine. Mitochondria function was checked using qPCR, Mitotracker DeepRed stain, and Seahorse MST assay.

### Correlative Light-Electron Microscopy

Sorted cells were fixed with 4% PFA and immobilized on Mattek gridded dishes (P35G-1.5-14-C-GRD, MatTek CorpAshland) using Cell Tak™ (Corning) and stained with Hoechst (1 μg/ml, Molecular Probes) for 20 min in PBS. Samples were acquired using an Olympus FluoVIEW FV3000RS confocal microscope with a UPLSAPO 60XS (NA 1.3) Silicone objective.

After acquisition of the fluorescence images and the grid reference coordinates, cells were fixed with 2.5% glutaraldehyde in 0.1 M cacodylate buffer pH 7.4 for 1 hr at room temperature. Sample were then postfixed in 1% osmium tetroxide, 1.5% potassium ferrocyanide in 0.1 M cacodylate buffer for 1 hr on ice and en-bloc stained in 0.5% uranyl acetate overnight at 4° C. Samples were dehydrated in increasing concentrations of ethanol and infiltrated in epoxy resin (Sigma-Aldrich). After curing at 60° C. for 48 hrs embedded cells were removed from the glass coverslips by dipping in liquid nitrogen. Ultrathin sections were obtained using an ultramicrotome (UC7, Leica microsystem, Vienna, Austria), collected on formvar carbon coated slot copper grids, stained with uranyl acetate and Sato's lead solutions and observed in a Transmission Electron Microscope Talos L120C (FEI, Thermo Fisher Scientific) operating at 120 kV. Images were acquired with a Ceta CCD camera (FEI, Thermo Fisher Scientific). TEM images were then aligned to fluorescence images using the ICY ec-CLEM plugin.

### Restriction Enzyme Analysis of mtDNA

Total DNA was isolated from sorted Mito+ and Mito− cells following co-culture of CD8+ T cells from Balb/c mice with Mito-DsRed labeled mouse DM5 BMSCs using QuickExtract™DNA Extaction Solution (Lucigen). Total DNA was also isolated from Mito-DsRed labelled mouse DM5 BMSCs and Balb/c CD8+ T cells as a control. A 385 bp fragment (9072-9456) of mitochondrial mt-Co3 gene containing the A9348G polymorphism site was amplified from the samples by PCR using a thermocycler (BioRad) with the following primers: mt-Co3-F, (CGAAACCACATAAATCAAGCCC; (SEQ ID NO. 9)) and mt-Co3-R (CTCTCTTCTGGGTTTATTCAGA; (SEQ ID NO. 10)). The PCR product was then digested with PflFI (New England Biolabs) that recognizes the Aspl restriction site for 15 mins at 37° C. and the fragments were visualized by electrophoresis in a 2% agarose gel containing 0.5 μg/ml EtBr.

### Mitochondria Stress Test Assay

A Seahorse XFe96 Analyzer (Agilent) was used to determine OCR for sorted Mito+ and Mito− CD8+ T cells. Sorted cells were washed in assay media [XF Base media (Agilent) with glucose (10 mM), sodium pyruvate (1 mM) and L-glutamine (2 mM) (Gibco), pH 7.4] at 37° C. before being plated onto Seahorse cell culture plates coated with Cell-Tak™ (Corning) at 2.5×10⁵ cells per well. After cell adherence and equilibration, OCR (pmol/min) was measured at steady state and after sequential injection of oligomycin (1.5 μM), BAM15 (2.5 μM), rotenone (1 μM) and antimycin A (1 μM) (Sigma-Aldrich). Experiments with the Seahorse system utilized the following assay conditions: 2 min mixture, 2 min wait, and 3 min measurement. SRC was calculated as oxygen consumption rate (OCR) at maximum rate (OCRMax)-OCR in basal state (OCRBas).

### Bulk RNA Sequencing

For human samples, total cellular RNA was isolated from Mito+ and Mito-CD8+ cells using the RNeasy Mini Kit (Qiagen). The concentration and quality of the purified RNA was analyzed using the RNA ScreenTape Kit (Agilent). Generation of dsDNA libraries for Illumina sequencing from total cellular RNA was carried out using the SMART-Seq Stranded Kit from Takara according to the manufacturer's instructions. The quality of dsDNA libraries was analyzed using the High Sensitivity D1000 ScreenTape Kit (Agilent) and concentrations were assessed with the Qubit dsDNA HS Kit (Thermo Fisher Scientific). Equimolar amounts of each human library were pooled and sequenced on a NextSeq 550 instrument controlled by the NextSeq Control Software (NCS) v2.2.0, using a 75 Cycles High Output Kit with a single index, and single-end (SE) run parameters. Image analysis and base calling were done by the Real Time Analysis Software (RTA, v2.4.11) at NGS-Core Facility (LIT). For mouse samples, total cellular RNA was isolated from post-sorted Mito+ and Mito− CD8+ cells using the RNeasy Plus Mini Kit (Qiagen) as per manufacturers recommended protocol. The quantity and quality of purified RNA was analyzed with the Ribogreen Assay (ThermoFisher Scientific) and HS RNA Fragment Analyzer chip (Agilent), respectively. Generation of libraries for Illumina sequencing was completed on these samples using KAPA HyperPrep kit (KAPA Biosystems). The quality and quantity of dsDNA libraries was analyzed using the DNA-5k Caliper (PerkinElmer) and Qubit dsDNA HS Kit (Thermo Fisher Scientific). Sequencing was carried out on the NovaSeq 6000 sequencer S4.

### Bioinformatic Analysis of bulkRNA Sequencing Data

For data analysis of RNA sequencing data, the output data from the NextSeq550, “.bcl” files, were converted into “.fastq” files with the bcl2fastq software (v2.20.0.422). For the human and mouse RNA Seq data, QC Analysis and read mapping was performed using the SnakePipes analysis pipeline (v2.5.1). The pipeline used among others using the following software: samtools (v1.9), STAR (2.7.4a) featureCounts (v2.0.0). Genome: GRCh38_gencode_release2 (ftp://ftp.ebi.ac.uk/pub/databases/gencode/Gencode_human/release_29/) and GRCh38.primary_assembly.genome.fa.gz). Mouse Genome GRCm38. primary_assembly (ftp://ftp.ebi.ac.uk/pub/databases/gencode/Gencode_mouse/release_M23/GRCm38.primary_assembly.genome.fa.gz). QC of the count matrix and DE-Gene calling was performed with DESeq2. Gene counts were prefiltered with edgeR: filterByExpr and the fdr was set to (Padj.hs<0.05) for the human data set. Volcano plots were produced with the enhancedVolcano and ggrepel package. For analysis of co-regulated and orthologous genes DESeq result tables were merged using orthologous annotation derived from ENSEMBL using the biomaRt package. Only orthologous genes were taken forward. Subsequently postively and negatively co-regulated genes were selected. The resulting gene table was selected for genes showing a baseMean.hs expression of 100 counts and Padj.hs<0.05. For the heatmap data was subset for human data to comply with baseMean.hs>100 and Padj.hs<0.05 and mouse data to show a p-value below 0.15. Variance stabilizing transformed (vst-function) expression data (DESeq2) was Z-score transformed for the mouse and human dataset separately. For hierachical clustering (euclidean distance, method complete) both data sets were combined and plotted with pheatmap. Over representation analysis of up/down regulated co-regulated genes was performed using EnrichR with genes complying with: human: baseMean.hs>100 and Padj.hs<0.05 (59 genes).

### CRISPR-Cas9 TLN2 Knockout

To knockout TLN2 or CD2 control genes in both human CD8+ T cells and Mito-DsRed BMSCs we used Lonza™ P3 Primary Cell 4D-Nucleofector™ X Kit S (Lonza™ V4XP-3032) and Lonza™ P1 Primary Cell 4D-Nucleofector™ X Kit S (Lonza™ V4XP-1032), respectively, with TrueCut™ Cas9 Protein v2 (ThermoFischer), Alt-R® CRISPR-Cas9 tracrRNA (IDT) and the predesigned crRNA TLN2-(AB & AC) and CD2-(AA, AD&AE) (IDT). In brief, tracrRNA (200 μM) and crRNA (200 μM) were mixed equimolar proportions (0.75 μl each with 1.5 μL IDTE Buffer, per guide reaction), incubated 5 mins at 95° C. in a ProFlex PCR System (Applied Biosystems™) and allowed to cool to RT for 20 mins. Newly formed gRNA was then combined with Cas9 enzyme (3 μL of gRNA and 1.2 μL of TrueCut Cas9 for each guide, topped up to 12.6 μL with IDTE Buffer) and incubated at RT for 20 mins to create TLN2 and CD2 RNP complexes. CD8+ T cells (1×10⁶, pre-activated for 24 hrs) and Mito-DsRed (5×10⁵, low confluence) were seeded in 20 μL of P3 and P1 electroporation buffer solution, respectively, as per manufacture's instructions in a 96-well round-bottom plate. TLN2 and CD2 RNP complexes were combined with cell suspensions in buffer solution and immediately transferred to a 16-well Nucleocuvette strip and the electroporation protocol CA137 (CD8+ T cells) or FF104 (MitoDsRed BMSCs) was performed on a Nucleofector Unit. After electroporation, warm media was added to the wells of the Nucleocuvette strip and cells were allowed to recover for 10 mins at RT, before being transferred back into tissue culture plates for expansion. Four days later human co-cultures were set up with either Mito-DsRed hBMSC or CD8+ T cells with TLN2 KO or CD2 KO (CRISPR control) and DsRed transfer rates to CD8+ T cells were analyzed using flow cytometry. Knockout efficiency was determined prior to co-culture using standard western blot. The protein was isolated using Pierce™ RIPA buffer (Thermo Scientific) and 20-30 μg of the protein from each sample was loaded into 4-20% Criterion™ TGX™ Precast Midi Protein Gel (BioRad) and gel electrophoresis was run for 45 mins. Protein was transferred to Trans-Blot Turbo Midi 0.2 μm PVDF Transfer membrane (BioRad) and stained with mouse anti-TLN-2 primary antibody (53.8, BIO-RAD) overnight, followed by washing and incubation with HRP-conjugated anti-Mouse IgG (Cell Signaling). Western blots were visualized using Pierce™ ECL Western Blotting Substrate (BioRad) on ChemiDoc Imaging Systems (Bio-Rad) at various exposure times.

### Inhibitor Drug Treatment Study

For pharmacological inhibition studies, both mouse and human Mito-DsRed BMSCs or CD8+ T cells were treated with 1-10 μM of farnesyltransferase/geranylgeranyltransferase 1 inhibitor (L-778123) (Biomol) incubated separately in their respective basal media for 7 hrs before or during co-culture. Cell viability of the different drug treatments was determined using the Trypan Blue exclusion test and quantified manually using a hemacytometer. All experiments were performed on at least 3-6 biological replicates/donors per condition.

### Murine Syngeneic Melanoma Model

C57BL/6 female mice ages 6-8 weeks were injected subcutaneously with 3×10⁵ B16KVP cells in 100 μl of PBS. On day 10 after tumor inoculation, host mice received 6Gy (C57BL/6 mice) or 2Gy (NCG mice) sub-lethal irradiation prior to transfer of 1×10⁵-1.25×10⁵ Mito+ and Mito [symbol] pmel-1 CD8+ T cells. An untreated group that received no adoptively transferred cells was also included as a control. To support viability and expansion of transferred CD8+ T cells mice received recombinant IL-2 intraperitoneally (2.4×10⁵ IU/day of rhIL-2 for 3 doses for 3 days). Mice were monitored thrice weekly for survival and tumor size using a caliper. The survival endpoint was reached when the mean diameter of the tumor size is 1.5 cm. For kinetic studies, tumors and spleens were collected on day 7 post adoptive transfer. Tumors were cut into two equal portions using a scalpel; one half of the tumors was embedded in Tissue-Tek O.C.T. compound (Sakura Finetek) and immediately frozen for immunohistochemistry, and the other portion of tumors were weighed, digested, and tumor-infiltrating lymphocytes were analyzed by flow cytometry.

### Frozen Immunohistochemistry

Frozen tissues were sliced to 5 μm thick sections in a cryostat and fixed with acetone for 10 min at −20° C., left to dry for 20 minutes and then washed three times with PBS. Sections were blocked with 2% rat serum for 45 mins prior to overnight incubation with anti-mouse Ly5.1 antibody (1:100 dilution) in a humidified chamber at 4° C. Following PBS wash, tissue sections were counterstained with Hoechst 33342 (Invitrogen™, 2 μg/ml) for 10 min at room temperature, washed with PBS again, and mounted with ibidi mounting medium (Ibidi GmbH) and a glass coverslip. Images of the whole tissue sections were acquired using the LAS X Navigator tile scanning function on a Stellaris 8 confocal microscope (Leica Microsystems). ImageJ (version 1.54) was used to overlay a pseudocolor heatmap of Ly5.1 fluorescence intensity on confocal images of Hoechst 33342 to show the relative localization of adoptively transferred CD8+ T cells in the tumor structure.

### Quantification and Phenotype of Adoptively Transferred CD8+ T Cells

Spleens were mechanically disrupted, passed through 40 μm strainers and treated for 2 min at room temperature with 1×ACK lysis buffer (Elabscience). Tumor tissue was mechanically disrupted in C-tubes using the mtumor program on a GentleMACS (Miltenyi Biotec) followed by digestion for 10 mins with DNase at 37° C. Tumor-infiltrating lymphocytes were enriched using Ficoll Paque Plus (Cytiva) at 400×g centrifugation for 30 mins at 18-20° C. Splenocytes and tumor-infiltrating lymphocytes were stained separately with flow cytometry antibodies. CountBright™ Plus Absolute Counting Beads (Invitrogen™) were added to each sample for absolute quantification of tumor-infiltrating lymphocytes. For intracellular staining, cells were fixed and permeabilized using the FoxP3 staining kit (eBioscience) following surface marker staining. Quantification of adoptively transferred CD8+ T cells was determined using Ly5.1 antibody and normalized based on counting beads, dilution factor, and tumor weights, where applicable.

### Human In Vitro Cytotoxicity Assay

For human in vitro cytotoxicity assays, Mito+, Mito−, Mito+ EtBr or Mito− EtBr T cells were co-incubated with target NALM6-GL leukemia cells at a 1:5 effector to target ratio (15,000:75,000) in 100 μL of AIMV or RPMI complete media in a 96 well plate (Corning). GFP fluorescence intensity of the tumor cells was measured every 2 hrs an Incucyte Live-Cell Analysis Instrument (Essen Bioscience). Green calibrated unit (GCU) per mm2/image was obtained using the Incucyte image software analyzer with a threshold adjustment of 100 GCU in the green channel.

### Humanized B-Cell Malignancy Model

NALM6-GL (8×10⁵) were injected intravenously into NXG host mice, followed 3 days later by the administration of 1.25×10⁵ CD19-CAR+ CD8+ T cells that either acquired donor mitochondria (Mito+), did not acquire donor mitochondria (Mito−), or were cultured alone (CD8 mono). Recombinant human IL-15 (NCI) was injected intraperitoneally every other day (1 μg per mouse). Tumor burden was measured using the IVIS-Lumina III In Vivo Imaging System (PerkinElmer). After 7 days, blood was collected from mice to confirm the adoptive transfer of CD8+ T cells and to assess relative levels of circulating NALM6-GL cells in the blood.

### Quantification and Statistical Analysis

Statistical analyses were conducted using Prism software v9.4 (GraphPad Software, La Jolla, California, USA). Spare respiratory capacity from Seahorse metabolic assay and inhibitor studies were analyzed using one-way ANOVA with Dunnett's multiple-comparison test. Transfer rates for TLN2 KO CRISPR editing are shown as mean±s.e.m. relative to CD2 KO CRISPR control and analyzed using unpaired two-tailed Student's t-test. Flow cytometry data were analyzed using unpaired two-tailed Student's t-test. Tumor growth curves and Incucyte cytotoxcity assay were analyzed using Wilcoxon Rank Sum test on the curve slopes. A log-rank (MantelCox) test was used for comparison of survival curves for pmel-1 melanoma and NALM6 B-cell malignancy in vivo models. The p value was denoted by * for p<0.05, ** for p<0.01, *** for p<0.001, and **** for p<0.0001.

### Example 2: Intercellular Nanotubes Enable Mitochondrial Trafficking From BMSCs to CD8+ T Cells

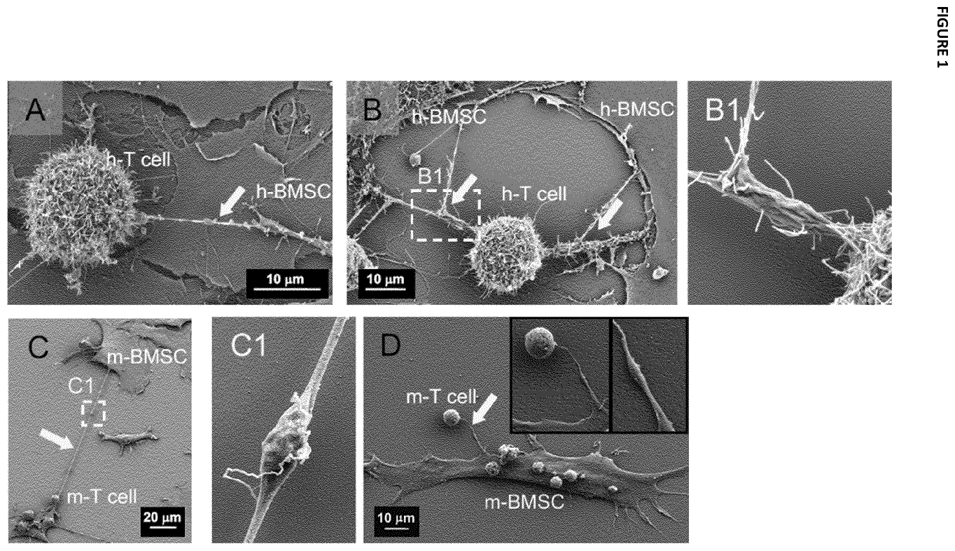

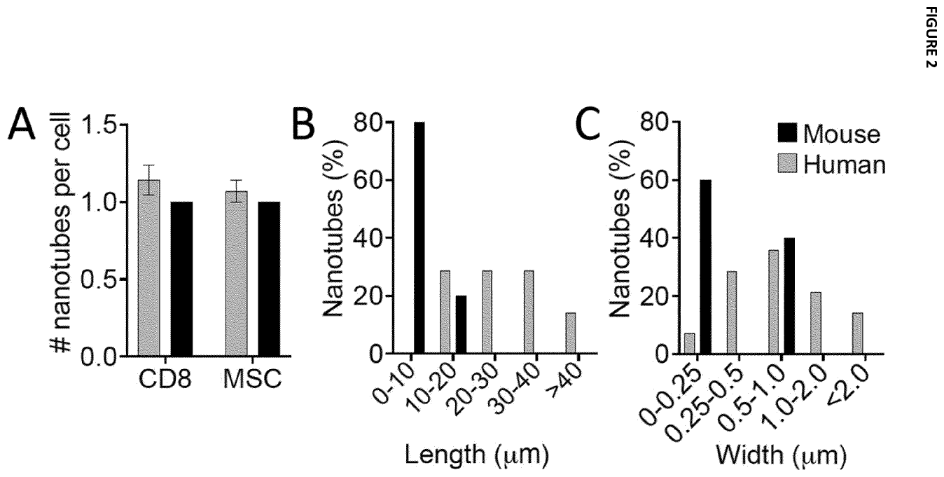

To investigate the interaction between BMSCs and CD8+ T cells, a co-culture study with either human or mouse BMSCs and species-matched CD8+ T cells on glass coverslips was set up. Cells were fixed after 24 hrs and examined using field-emission scanning electron microscopy (FESEM). Complex nanotube structures were frequently found which physically bridge BMSCs and CD8+ T cells in both human (FIGS. 1A-1B) and mouse (FIGS. 1C-1D) settings. The detection of complex branching nanotubes indicates that these intercellular bridging structures were not mere stress fibers (FIG. 1B, B1). The average number of nanotubes per cell for both BMSCs and CD8+ T cells was one per cell (FIG. 2A). However, as nanotubes are ultrafine structures that may be lost during sample processing, the actual number per cell may be higher. Overall, mouse nanotubes were shorter (<20 μm) and narrower (<1 μm) compared to their human counterparts whose dimensions were more variable with maximal lengths and widths exceeding 40 μm and 2 μm, respectively (FIGS. 2B-2C). Interestingly, despite the narrow average width, some nanotubes exhibited enlarged segments, which may accommodate trafficking organelles, such as mitochondria (FIGS. 1C1 and D, far right inlet).

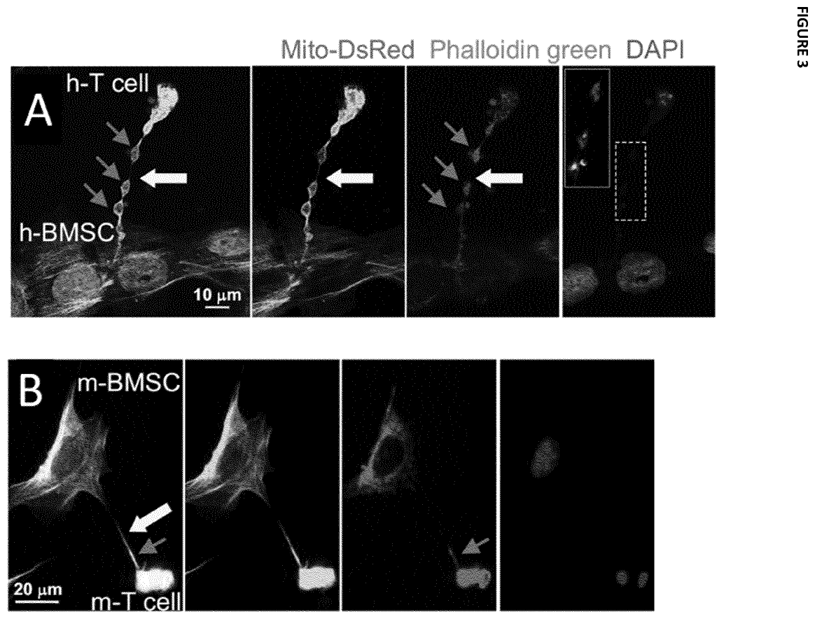

To determine if mitochondria are indeed transported within nanotubes from BMSCs to CD8+ T cells, BMSCs were transduced with a DsRed-tagged mitochondrial subunit protein (COX8A), known as Mito-DsRed, and evaluated BMSC-CD8+ T cell co-cultures using confocal microscopy. After 24 hr co-incubation, it was observed that a fraction of CD8+ T cells acquired DsRed signal in both human (FIG. 3A) and mouse (FIG. 3B) settings. Phalloidin Green was used to stain F-actin of both BMSCs and CD8+ T cells to delineate nanotube structures. Enlarged portions of the nanotubes were observed (FIG. 3A) confirming the FESEM observations. Notably, also DsRed signals were detected co-localized within these regions (FIG. 3A) substantiating that TNTs serve mitochondrial trafficking from BMSCs to CD8+ T cells. Interestingly, high magnification of TNT bulges and locally adapted exposure of DNA-specific DAPI staining showed traces of DNA that are co-localized with DsRed pockets, consistent with the presence of intact mitochondria (FIG. 3A, inlet). Taken together, FESEM and confocal studies revealed nanotube-mediated intercellular trafficking of mitochondria from BMSCs to CD8+ T cells in both mouse and human cells.

### Example 3: Establishment and Validation of Mitochondrial Transfer as Technology Platform

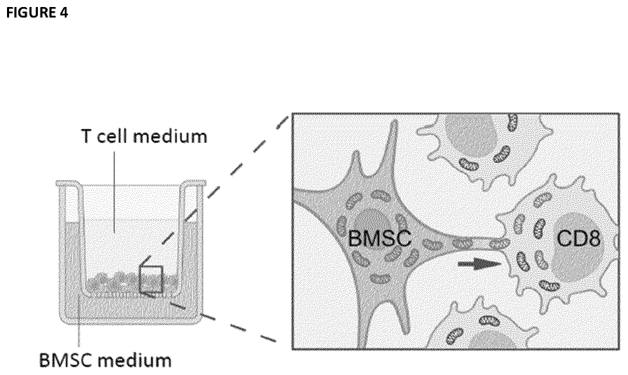

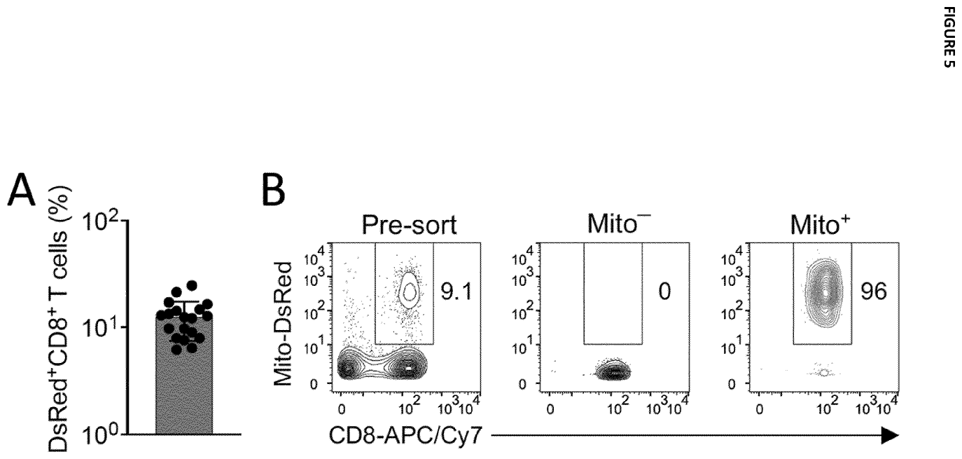

Next it was tested if a co-culture transwell system (FIG. 4) could improved cell viability and transfer rates compared to conventional 2D tissue culture plastic. It was found that cell viability and transfer rates of DsRed mitochondria from BMSCs to CD8+ T cells indeed increased (data not shown). Hereafter, CD8+ T cells that acquire donor mitochondria in co-culture will be referred to as Mito+, whereas CD8+ T cells that do not will be referred to as Mito− cells. The average percentage of Mito+ (DsRed+CD8+) cells after a 36 hr co-culture ranged from 6.2% to 24.7% with a mean of 12.5% (FIG. 5A). Mito+ and Mito− cells could be sorted to purities higher than 90% (FIG. 5B).

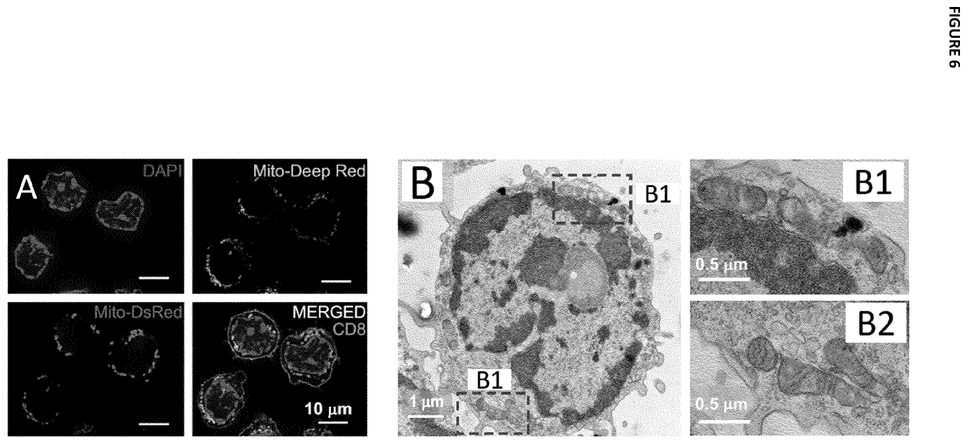

To validate the transfer of mitochondria, confocal microscopy of sorted Mito+ cells was performed. Sorted Mito+ cells were stained with a fluorescently conjugated CD8-specific antibody to delineate the cell membrane and MitoTracker Deep Red to determine if mitochondria had active membrane potential. It was found that DsRed mitochondria were internalized within recipient CD8+ T cells and maintained intact mitochondrial membrane potential (FIG. 6A). To more precisely attribute DsRed signals to mitochondria, correlative light-electron microscopy (CLEM) was used, which employs a combination fluorescence microscopy with an electron microscope, to image Mito+ cells. It was found that DsRed signals co-localized with mitochondria structures and were not diffuse in the cell cytosol (FIG. 6B). Qualitative analysis showed organized cristae and double membrane arrangements typical of a normal mitochondria phenotype (FIGS. 6B1 and 6B2).

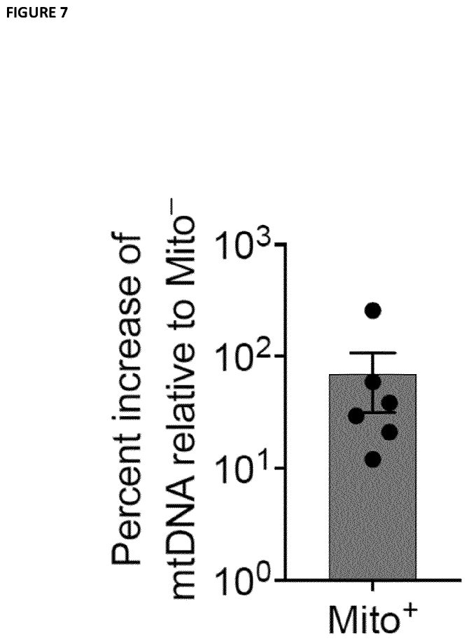

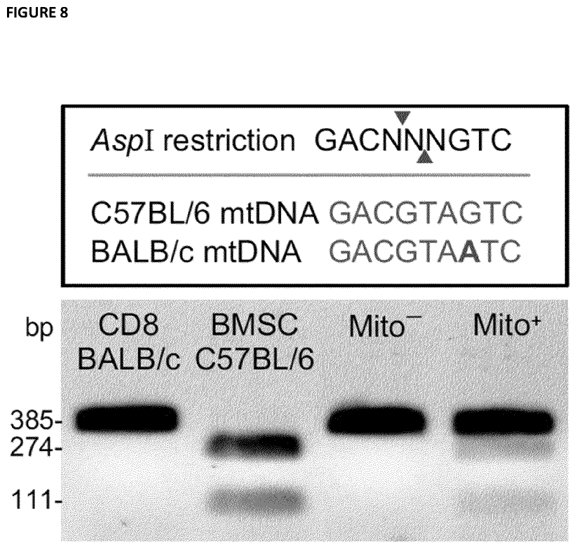

To assess if the transferred donor mitochondria increased the overall mitochondrial mass of the recipient CD8+ T cells, quantitative real-time PCR was used to determine mtDNA content of Mito+ and Mito− cells as measured by mt-Co2 gene normalized to the nuclear gene standard reference gene App. Mito+ cells showed an increase in mtDNA content ranging from 12% to 256% (median=33.84%) compare to Mito− cells (FIG. 7). To validate if the mitochondrial transfer from BMSCs contributes to this increase in mitochondrial content, the Mito-DsRed BMSC line, which was derived from a C57BL/6 mouse strain, was co-cultured with CD8+ T cells isolated from BALB/c mice. BALB/c mtDNA has a single nucleotide polymorphism at A9348 in the mt-Co3 gene that disrupts an Aspl restriction site that is normally present in C57BL/6 mtDNA allowing genetic discrimination of endogenous and transferred mitochondria. mtDNA was extracted from highly enriched Mito+ and Mito− cells (>98% purity) and restriction enzyme analysis of the mt-Co3 target region was performed, as previously described (Nucleic acids research (2003) 31, 5349-5355). Mito− cells showed a uniform 385 bp band, congruent with non-co-cultured BALB/c CD8+ control cells. Strikingly, Mito+ cells showed a mixture of BALB/c and C57BL/6 mtDNAs demonstrating the presence of both endogenous and donor mtDNA (FIG. 8).

### Example 4: BMSC Mitochondrial Transfer Enhances CD8+ T Cell Metabolic Fitness

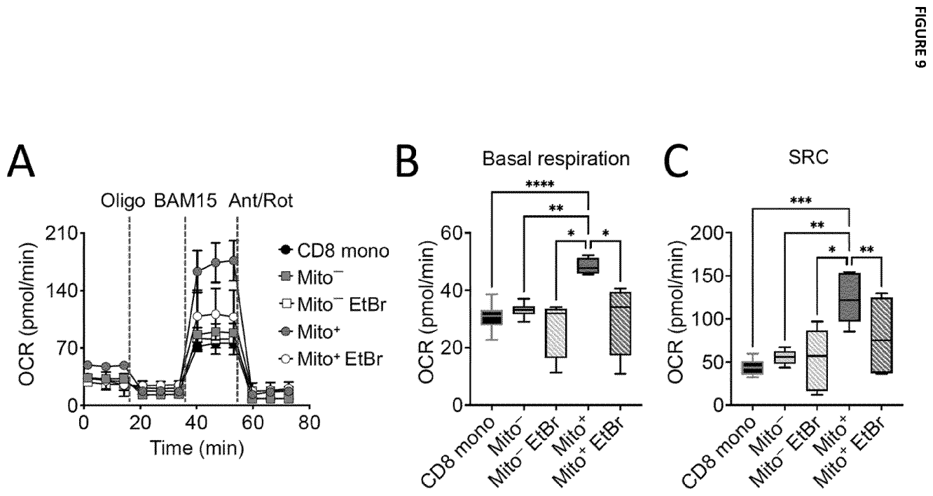

A key task of mitochondria is the generation of ATP, primarily via aerobic respiration. To evaluate the effect of donor mitochondria on CD8+ T cell respiration, oxygen consumption rates were measured at a steady state and after perturbation with diverse modulators of mitochondrial respiration (FIG. 9A). Overall, Mito+ cells showed significantly higher basal respiration and spare respiratory capacity (SRC) compared to either Mito− cells or CD8+ T cells that were not co-cultured with BMSCs (CD8 mono) (FIGS. 9B-C). To exclude the possibility that the enhanced mitochondrial activity of Mito+ cells was due to the intercellular transfer of other cytoplasmic factors, additional control groups were set up using Mito-DsRed-labeled BMSCs whose mitochondria were selectively rendered dysfunctional by pre-treatment with low-dose Ethidium Bromide (EtBr). EtBr-treated BMSCs had lower mitochondrial membrane potential, decreased mtDNA, and exhibited severely impaired mitochondrial respiration (data not shown). However, these EtBr-treated BMSCs maintained their capacity to transfer DsRed-labeled mitochondria to recipient CD8+ T cells (Mito+ EtBr). Notably, the improvement in mitochondrial fitness observed in Mito+ cells was abrogated in Mito+ EtBr cells (FIGS. 9A-C). Altogether, these results not only functionally validate the effective transfer of mitochondria to CD8+ T cells but also demonstrate that the increased metabolic activity in Mito+ cells is donor mitochondria-dependent.

### Example 5: Mitochondrial Transfer Between BMSCs and T Cells Depends on Talin-2

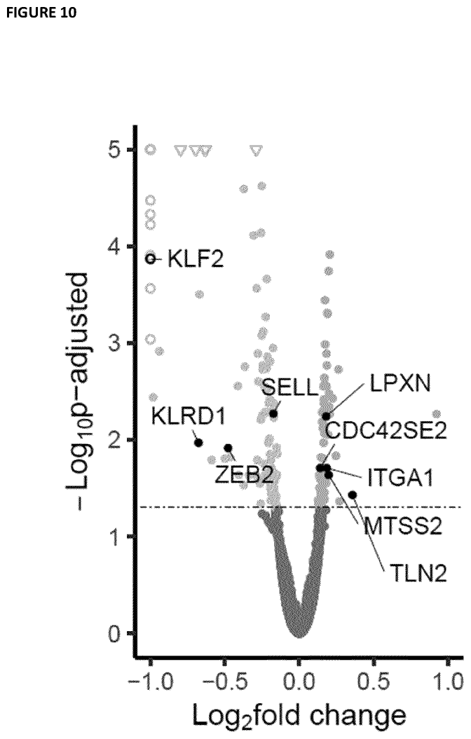

Nanotubes are unique cell protrusions and undergo several phases of development, including; (i) initiation of membrane curvature via inverse BAR (I-BAR) proteins (ii) extension of the membrane protrusion via actin polymerization and integrin binding mediated by focal adhesion proteins (FAP), until (iii) reaching the adjacent cell and undergoing membrane fusion to complete the intercellular connection (Trends in Cell Biology (2021) 31, 130-142). Rho-GTPases have been shown to play a key role throughout this process from activating I-BAR proteins to regulating focal adhesion and assisting in trafficking mitochondria through nanotubes (The EMBO Journal (2014) 33, 994-1010; Biochem Biophys Res Commun (2010) 401, 527-532). To further unravel the mechanisms governing mitochondrial transfer, RNA-sequencing on sorted human and mouse Mito+ and Mito− cells was performed. It was found that several molecules involved in membrane curvature initiation, protrusion, and elongation were more strongly expressed in human Mito+ cells, including MTSS I-BAR Domain Containing 2 (MTSS2, also known as ABBA-1), which regulates plasma membrane dynamics and Rho GTPase activity, Talin-2 (TLN2), a cytoskeletal protein involved in actin filaments assembly, which mediates their interaction with integrins and membrane protrusions, Leupaxin (LPXN), a focal adhesion-associated protein, Integrin alpha-1 (ITGA1) involved in CD8+ T cell motility, and CDC42 Small Effector 2 (CDC42SE), a downstream regulator of small Rho-GTPase CDC42 involved in actin assembly and cell shape, were all upregulated in human Mito+ cells (FIG. 10). The top 22 co-regulated genes were TLN2, LPXN, LNPEP, CCNG1, MVB12B, RASGRP2, HGS, CALCOCO1, RIPOR2, RASA3, SAMD3, TSPAN32, SELL, PAN2, SEC31B, NFATC2IP, MT-ND4, CDC25B, MT-CO1, KLRD1, KLF2 and INF2.TLN2 and LPXN were among the genes upregulated in Mito+ cells in both mouse and human settings. Consistent with this observation, Gene Ontology (GO) analysis showed that focal adhesion and cellular substrate junction genes were among the gene sets most significantly enriched in co-regulated genes.

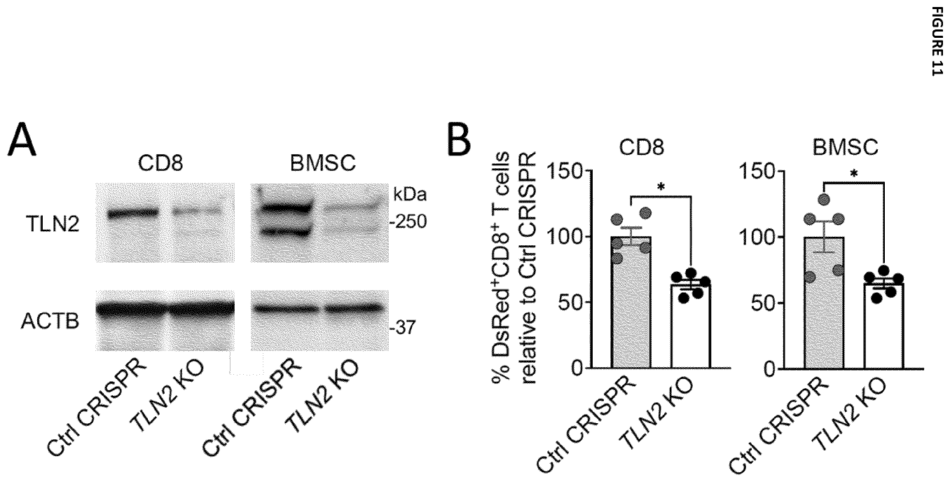

The heightened expression of genes involved in the formation of membrane protrusions and extensions suggested that CD8+ T cells uptaking BMSC mitochondria display an increased ability to establish TNT connections. Using L-788,123, an inhibitor of farnesyltransferase and geranylgeranyltransferase type 1 that has been previously shown to partially block nanotube-mediated mitochondria transfer (Nature Nanotechnology (2022) 17, 98-106), it could be confirmed that TNTs is a primary mediator of mitochondrial transfer in both our mouse and human co-culture setups (data bot shown). Further studies were the focused on TLN2 given that it was the second highest upregulated gene in human Mito+ cells and was similarly regulated in mouse cells that acquired donor mitochondria. It was speculated that TLN2 may play a role in the extension of nanotubes and subsequent mitochondrial transfer. To test this hypothesis, the CRISPR/Cas9 gene editing technology was used to knock out TLN2 in CD8+ T cells as well as in Mito-DsRed BMSCs prior to co-culture. Although complete loss of TLN2 expression was observed (FIG. 11A), the reduction of TLN2 levels in both CD8+ T cells and BMSCs was sufficient to significantly impair mitochondrial transfer rates (FIG. 11B). These findings implicate TLN2 as a key regulator of nanotube-mediated mitochondrial transfer.

### Example 6: Mitochondrial Transfer Enhances CD8+ T Cell Antitumor Immunity Against Solid Tumors

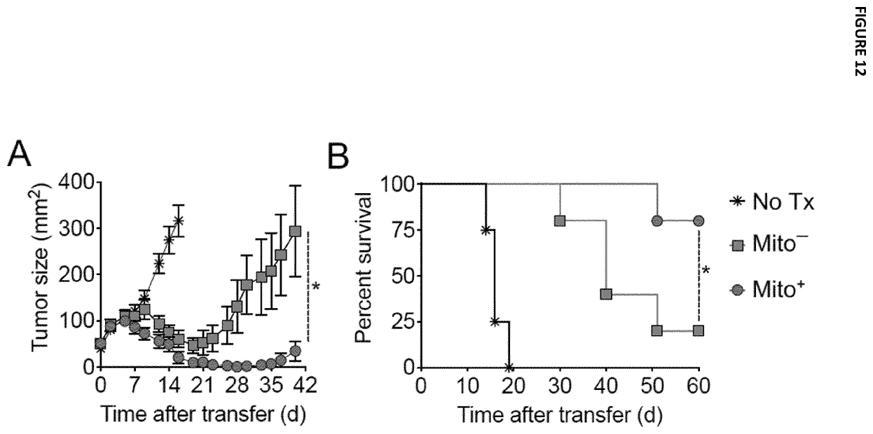

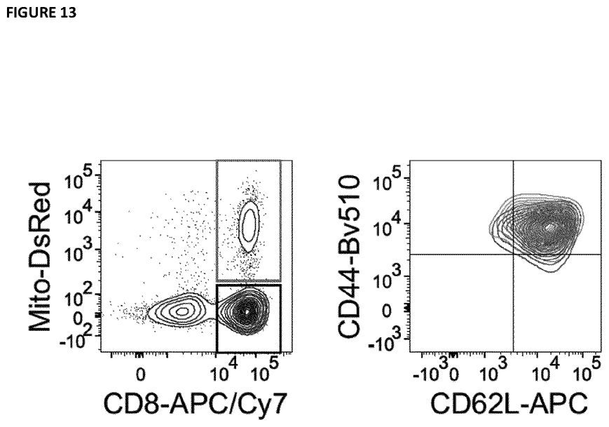

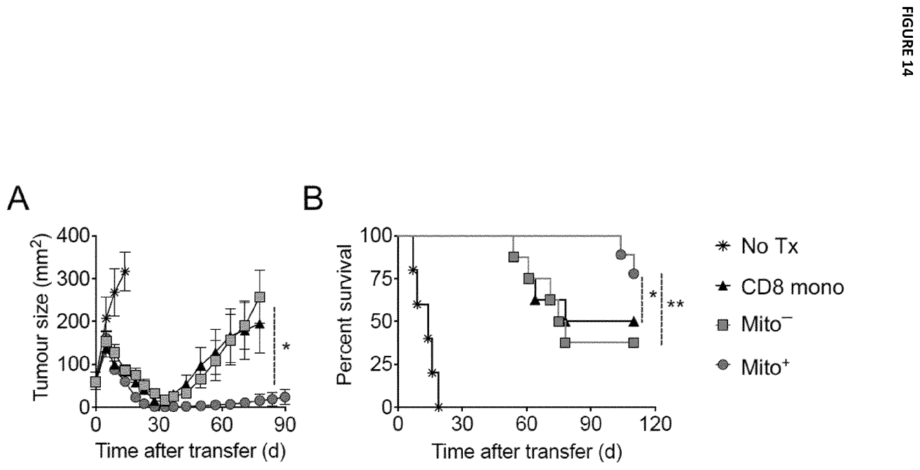

It was hypothesized that the higher SRC observed in Mito+ cells would provide the energetic advantage to thrive in harsh microenvironments, such as tumors, and additionally compensate for any loss of mitochondria to cancer cells that would have led to a loss of T cell viability. Mito+ and Mito− pmel-1 CD8+ T cells were generated, which express a transgenic T cell receptor (TCR) recognizing the melanoma antigen, gp100, and transferred them into irradiated mice bearing subcutaneous B16KVP melanoma (JCI Insight (2019) 4 (10): e124405). Strikingly, Mito+ cells mediated a more robust tumor regression compared to Mito− cells (FIG. 12A), significantly prolonging mouse survival (FIG. 12B). Importantly, the enhanced antitumor responses mediated by Mito+ cells were not due to a skewing of mitochondria uptake by CD8+ T cell subsets (FIG. 13). Moreover, similar results were observed using immunodeficient NCG mice as tumor-bearing hosts, ruling out the contribution of endogenous immune cells to the observed therapeutic outcome (FIG. 14).

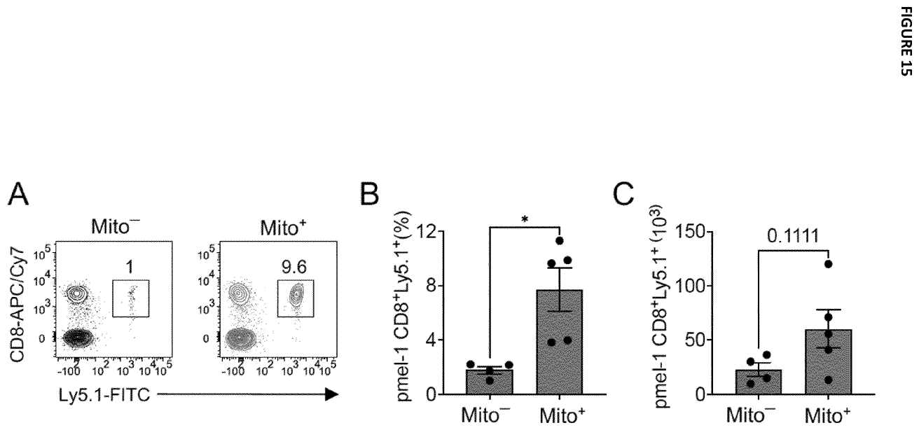

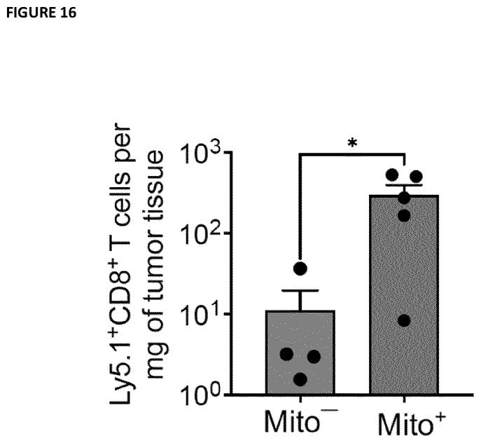

To gain further insight into the cellular mechanisms behind the augmented antitumor efficacy of Mito+ cells, Mito+ and Mito− pmel-1 CD8+ T cells carrying the Ly5.1 congenic marker were administered to enable tracking of transferred cells into tumor-bearing wild-type mice. Seven days after adoptive transfer, increased frequencies and numbers of pmel-1 cells in the spleens of mice were found that received Mito+ cells, indicating that transferred mitochondria confer more robust cell engraftment and expansion (FIGS. 15A-C). Similarly, we measured higher numbers of pmel-1 cells in tumors harvested from Mito+ cell-treated mice (FIG. 16). Notably, Mito+ cells infiltrated tumors efficiently whilst Mito [symbol] T cells were excluded from tumors and largely confined at the tumor periphery. Interestingly, DsRed-labeled mitochondria could still be detect within tumor-infiltrating Mito+Ly5.1+cells (data not shown).

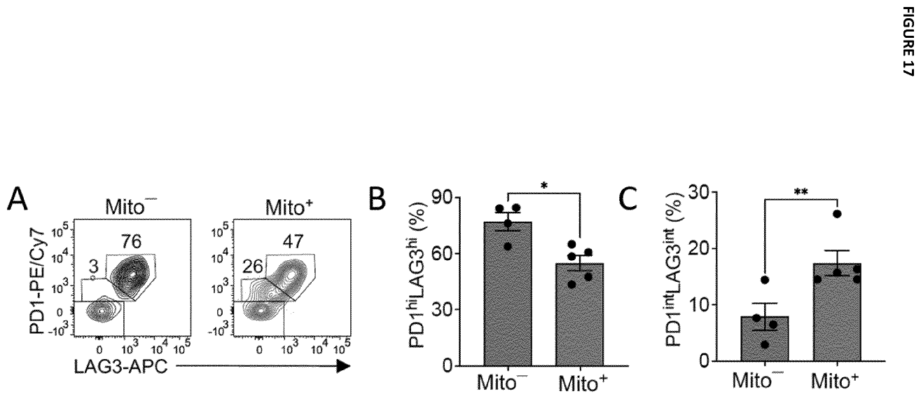

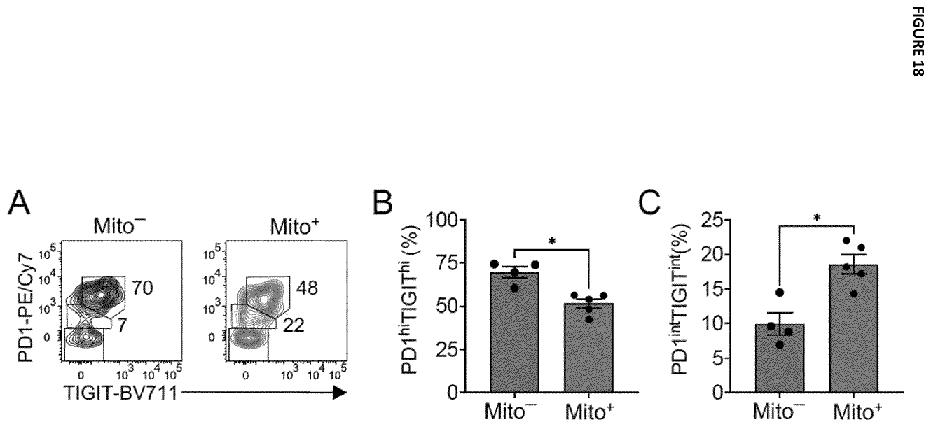

Next it was determined whether mitochondrial transfer conveys resistance to terminal exhaustion while supporting the differentiation of highly functional effectors. A high-dimensional analysis using an unsupervised clustering algorithm on the merged datasets in FlowJo (Cytometry A (2015) 87, 636-645) was performed. FlowSOM identified three different putative pmel-1 T cell subpopulations (data not shown). Mito+ cells were enriched in Cluster 1, which is characterized by low-intermediate expression of co-inhibitory receptors, including Programmed Cell Death Protein 1 (PD1) and lymphocyte-activation gene 3 (LAG3), whereas Mito-cells were enriched for Cluster 3, which is marked by low expression of the cytotoxic molecule, Granzyme B (Gzmb) (data not shown). Consistent with these observations, higher frequencies of terminally exhausted PD1^{hi}LAG3^{hi} in Mito− compared to Mito+ cells was found, which instead contained a larger population of cells with intermediate levels of these inhibitory receptors (FIGS. 17A-C). Similarly, TIGIT expression was lower in Mito+ cells, further supporting the idea that donor mitochondria promote resistance of CD8+ T cells to terminal exhaustion (FIGS. 18A-C). In line with this view, there was an inverse correlation (R=−0.54, p=0.0376) between the percentage of DsRed-retaining cells and the levels of PD1 in intratumoral pmel-1 cells (data not shown).

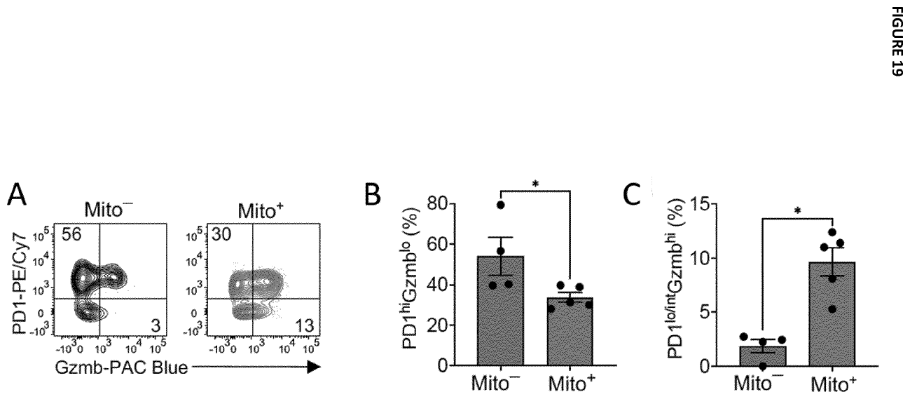

Flow cytometry analyses also revealed a significantly higher portion of PD1^{low/int}Gzmb^{hi} cells in the Mito+ group compared to Mito− cells (FIGS. 19A-C), indicating that mitochondria-boosted CD8+ T cells are more functionally active in the tumor microenvironment. These data were further supported by the detection of higher frequencies of terminally-differentiated effector cells (KLRGhiILR7Rαlow) in Mito+ cells, which are necessary for tumor killing and clearance (data not shown). Overall, these results demonstrate that the acquisition of donor mitochondria by CD8+ T cells provides significant advantages in terms of cell expansion, tumor penetration, resistance to exhaustion, and differentiation into highly functional killers.

### Example 7: Mitochondrial Transfer Enhances Human CD19-CAR CD8+ T Cell Antitumor Immunity

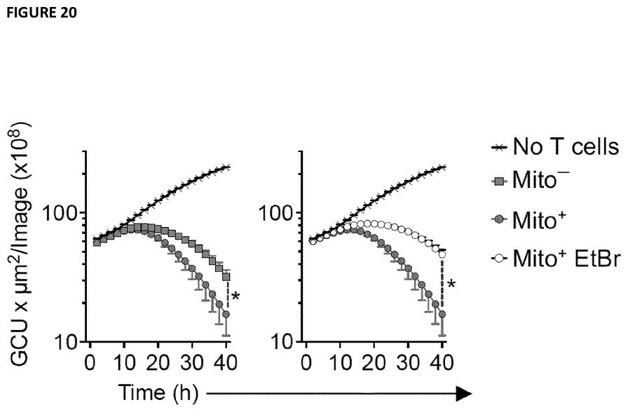

To assess whether mitochondrial transfer could also improve human antitumor T cell efficacy, human CD8+ T cells were transduced with a retroviral construct encoding a CD19-CAR and co-cultured them with Mito-DsRed BMSCs. Initially the cytotoxic capacity of CD19-CAR T cells in vitro against GFP-expressing NALM/6, an aggressive CD19+ human B-cell lymphoblastic leukemia (B-ALL) cell line (Int J Cancer (1979) 23, 174-180) was evaluated. Compared to Mito− cells, Mito+ cells demonstrated significantly increased tumor-killing capacity (FIG. 20). Strikingly, transfer of dysfunctional mitochondria (Mito+ EtBr) did not enhance antitumor activity, again demonstrating that the superior functionality observed in Mito+ cells is driven by donor mitochondria.

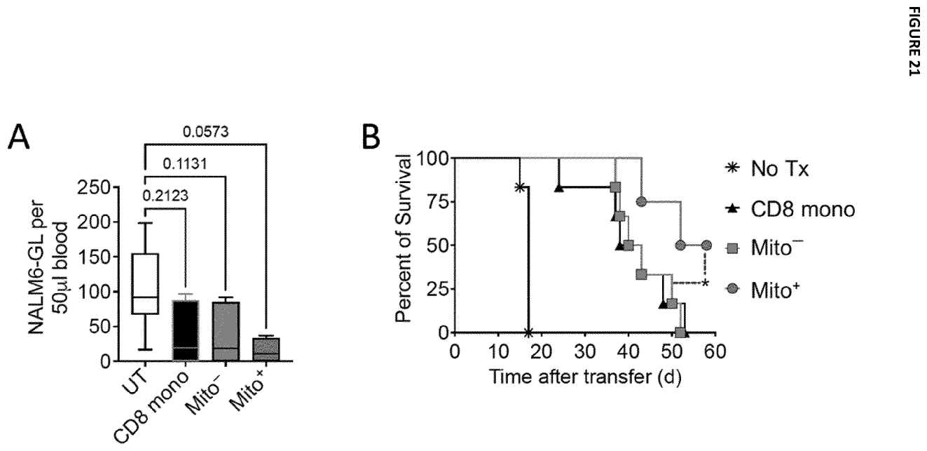

Next, we assessed the antitumor efficacy of CD19-CAR T cells in vivo, by transferring cells into NXG mice bearing systemic acute lymphoblastic leukemia (Blood (2016) 128, 519-528). Seven days after transfer, a lower number of circulating leukemic cells in mice receiving CD19-CAR Mito+ cells was detected compared to the untreated group (p=0.0573), whereas transferring Mito-cells or CD8 monocultured had only a minor impact on tumor burden in the blood (FIG. 21A). Mito+ cell enhanced antitumor activity resulted in prolonged control of NALM6 leukemia and significantly increased mouse survival compared to both Mito-and conventional monocultured CAR CD8+ T cells (FIG. 21B). Taken together, these findings highlight the translational potential of the mitochondrial transfer technology platform.

1. A method of augmenting mitochondria in mammalian CD8-positive T cells by culturing said CD8-positive T cells with mitochondria donor cells.

2. The method according to claim 1, wherein said mitochondria donor cell is a hematopoietic cell or a stem cell.

3. The method according to claim 1, wherein said mitochondria donor cell is a bone marrow stromal cell or a mesenchymal stem/stromal cell.

4. The method according to claim 1, wherein said mammalian CD8-positive T cells and/or said mitochondria donor cells are Talin-2 positive.

5. The method according to claim 4, wherein said mammalian CD8-positive T cells and/or said mitochondria donor cells are engineered to express Talin-2.

6. The method according to claim 1, wherein said mammalian CD8-positive T cells further comprises an antigen-specific receptor.

7. The method according to any one of claim 6, wherein said antigen-specific receptor is a T cell receptor (TCR) or a chimeric antigen receptor (CAR).

8. A mitochondria-augmented mammalian CD8-positive T cell obtained by the method of claim 1.

9. A mitochondria-augmented mammalian CD8-positive T cell.

10. A mitochondria-augmented mammalian CD8-positive T cell according to claim 9, wherein said mammalian CD8-positive T cells further comprises an antigen-specific receptor.

11. A mitochondria-augmented mammalian CD8-positive T cell according to claim 10, wherein said antigen-specific receptor is a T cell receptor (TCR) or a chimeric antigen receptor (CAR).

12. A method for treatment of cancer in a patient, said method comprising administering to the patient a mitochondria-augmented mammalian CD8-positive T cell according to claim 9.

13. The method of claim 12, wherein said cancer is a solid cancer or a hematological cancer.

14. A method for enhancing CD8-positive T cell antitumor immunity in a patient, said method comprising administering to the patient a mitochondria-augmented mammalian CD8-positive T cell according to claim 9.

15. The method according to claim 12, wherein said treatment additionally comprises an immune checkpoint inhibitor.

16. The method according to claim 14, wherein said treatment additionally comprises an immune checkpoint inhibitor.

17. A mitochondria-augmented mammalian CD8-positive T cell according to claim 8, wherein said mammalian CD8-positive T cells further comprises an antigen-specific receptor.

18. A mitochondria-augmented mammalian CD8-positive T cell according to claim 17, wherein said antigen-specific receptor is a T cell receptor (TCR) or a chimeric antigen receptor (CAR).

19. A method for treatment of cancer in a patient, said method comprising administering to the patient a mitochondria-augmented mammalian CD8-positive T cell according to claim 8.

20. A method for enhancing CD8-positive T cell antitumor immunity in a patient, said method comprising administering to the patient a mitochondria-augmented mammalian CD8-positive T cell according to claim 8.
